(19) United States
(12) Patent Application Publication
(10) Pub. No.: US 2022/0079944 A1
(43) Pub. Date: Mar. 17, 2022

(54) 2H-INDAZOLE DERIVATIVES AS THERAPEUTIC AGENTS FOR BRAIN CANCERS AND BRAIN METASTASES

(71) Applicant: BETA PHARMA, INC., Wilmington, DE (US)

(72) Inventors: Michael Nicholas GRECO, Lansdale, PA (US); Michael John COSTANZO, Warminster, PA (US); Jirong PENG, Mequon, WI (US); Don ZHANG, Princeton, NJ (US)

(21) Appl. No.: 17/426,296

(22) PCT Filed: Jan. 28, 2020
(86) PCT No.: PCT/US2020/015398

§ 371 Date: Jul. 28, 2021

Related U.S. Application Data
(60) Provisional application No. 62/798,220, filed on Jan. 29, 2019.

(51) Int. Cl. A61K 31/506; A61K 45/06; A61P 35/00

(52) U.S. Cl. CPC A61K 31/506; A61P 35/00; A61K 45/06

(57) ABSTRACT

Methods are disclosed for treating brain cancers or brain metastases from other cancers, or prevention of brain metastases, associated with CDK4 and/or CDK6 activities, where the methods comprise administering to a subject in need thereof a therapeutically effective amount of a compound of formula (I). Use of a compound of formula (I) for the manufacture of a medicament for treatment of brain cancer or brain metastases from other cancers, or prevention of brain metastases, associated with CDK4 and/or CDK6 activity is also disclosed.

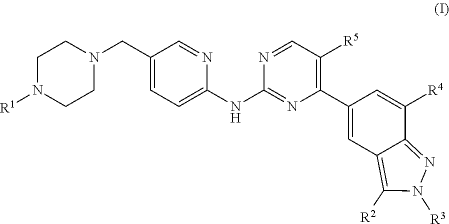

### CROSS-REFERENCE TO RELATED APPLICATIONS

This application claims priority under 35 U.S.C. § 119(e) to U.S. Provisional Application Ser. No. 62/798,220, filed on Jan. 29, 2019, the disclosure of which is incorporated herein by reference in its entirety.

### FIELD OF THE INVENTION

This application relates to a method of treating brain cancers and brain metastases using 2H-indazole derivatives and compositions thereof.

### BACKGROUND OF THE INVENTION

Cyclin-dependent kinases are a family of protein kinases that regulate cell division and proliferation. Cell cycle progression is controlled by cyclins and their associated cyclin-dependent kinases, such as CDK1-CDK4 and CDK6, while other CDKs such as CDK7-CDK9 are critical to transcription. CDK binding to cyclins forms heterodimeric complexes that phosphorylate their substrates on serine and threonine residues, which in turn initiates events required for cell-cycle transcription and progression (Malumbres, et al., Trends Biochem. Sci. 2005, 30, 630-641). Since uncontrolled cell proliferation is a hallmark of cancer, and most cancer cells exhibit deregulation of CDKs, inhibition of CDKs has emerged as a potential treatment for various cancers. Inhibitors with varying degrees of selectivity for CDKs have been reported. Selective CDK4/6 inhibitors are currently viewed as a promising class of potential cancer therapeutic agents due to the critical role of CDK4/6 in regulating cell proliferation and the toxic effects associated with inhibition of other CDKs.

Abemaciclib, palbociclib, and ribociclib are CDK4/6 inhibitors that have been approved recently for the treatment of HR⁺/HER2⁻ breast cancer.

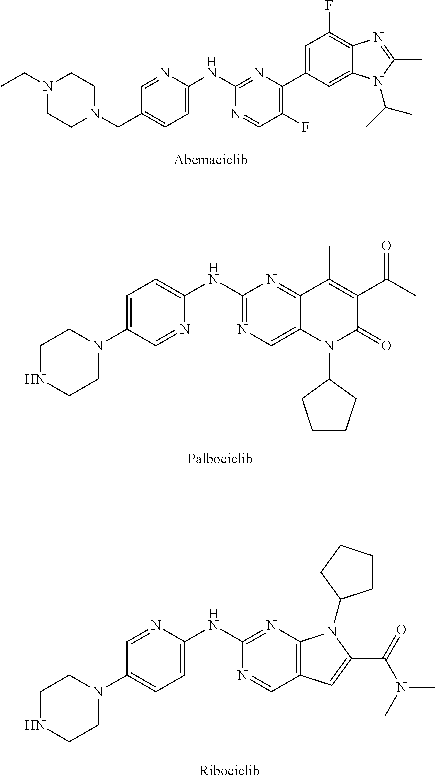 [SMILES: CCN1CCN(Cc2ccc(Nc3ncc(F)c(-c4cc(F)c5nc(C)n(C(C)C)c5c4)n3)nc2)CC1 ; CC(=O)c1c(C)c2nc(Nc3ccc(N4CCNCC4)cn3)ncc2n(C2CCCC2)c1=O ; CN(C)C(=O)c1cc2cnc(Nc3ccc(N4CCNCC4)cn3)nc2n1C1CCCC1]

However, none of these agents displays favorable blood brain barrier (BBB) permeability in pre-clinical pharmacokinetic (PK) and efficacy models. See, e.g., Raub, T. J. et al., Drug Metab. Dispos. 2015, 43, 1360-1371. Furthermore, both palbociclib and abemaciclib are p-glycoprotein (P-gp) substrates, a highly undesirable property for a potential CNS drug, and one that can preclude its development for diseases of the brain.

Brain metastases (or “secondary brain tumors”) refer to cancer cells that spread to the brain from the original diseased organs in the body, which can take place for any cancer, though more commonly from lung, breast, colon, kidney and melanoma. According to the literature, brain metastases occur in an estimated 24-45% of all cancer patients in the United States (see https://emedicine.medscape.com/article/1157902-overview), and in 10 to 30 percent of adult cancer patients (see https://www.mayoclinic.org/diseases-conditions/brain-metastases/symptoms-causes/syc-20350136). Brain metastases create pressure on the surrounding brain tissue and can cause various signs and symptoms, including severe pain. Treatment of brain metastasis would not only be instrumental to extending the lifespan of cancer patients, but also important to help reduce pain and other symptoms, thus improving the patients' life quality.

Thus, there is a clear unmet medical need to develop a CDK4/6 inhibitor with high BBB permeability.

### SUMMARY OF THE INVENTION

The present invention is based on the surprising discovery that indazole compounds of formula (I) are potent, selective CDK4/6 inhibitors that possess good blood brain barrier (BBB) permeability. Therefore, these compounds are useful therapeutic agents for the treatment or prevention of brain cancers and brain metastases from various other cancers.

In one aspect, the present invention provides a method of treating a brain cancer or brain metastases in a subject, the method comprising administration of a therapeutically effective amount of a compound of formula (I):

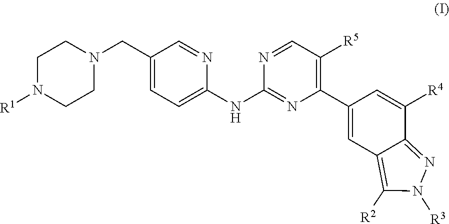

or a pharmaceutically acceptable salt, solvate, or prodrug thereof, wherein:

R¹ is hydrogen, C₁-C₈ alkyl, C₃-C₇ cycloalkyl, R⁶C(O)—, or R⁷O(CO)—;

R² and R³ are each independently hydrogen, C₁-C₈ alkyl, C₃-C₇ cycloalkyl, or C₃-C₇ cycloalkylmethyl;

R⁴ is hydrogen, halogen, C₁-C₈ alkyl, or C₃-C₇ cycloalkyl;

R⁵ is hydrogen or halogen;

R⁶ is hydrogen, C₁-C₈ alkyl; or C₃-C₇ cycloalkyl; and

R⁷ is C₁-C₈ alkyl; or C₃-C₇ cycloalkyl,

wherein any said alkyl or cycloalkyl is optionally substituted.

In another aspect, the present invention provides use of a compound of formula (I) in the manufacture of a medicament for the treatment of a brain cancer or brain metastases associated with CDK4 and/or CDK6 activity.

Compound 1, N-(5-((4-ethylpiperazin-1-yl)methyl)pyridin-2-yl)-5-fluoro-4-(3-isopropyl-2-methyl-2H-indazol-5-yl)pyrimidin-2-amine, is an example of a compound of formula (I), where R¹ is ethyl, R² is isopropyl, R³ is methyl, R⁴ is hydrogen and R⁵ is fluoro. Compound 1 is a potent, selective inhibitor of CDK4/6, useful in the treatment or prevention of diseases, disorders, or medical conditions mediated through certain CDKs, in particular CDK4 and CDK6, such as various types of cancers and inflammation-related conditions. Brain cancers, such as glioblastoma, represent a therapeutic area where a CDK4/6 inhibitor is anticipated to have a high potential for efficacy.

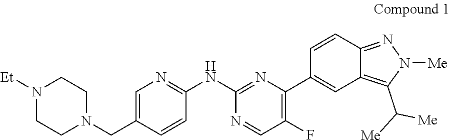 [SMILES: CCN1CCN(Cc2ccc(Nc3ncc(F)c(-c4ccc5nn(C)c(C(C)C)c5c4)n3)nc2)CC1]

In particular, the present invention provides methods of treating brain metastases of various cancers, including but not limited to breast cancers, lung cancers, especially non-small cell lung cancer (NSCLC), colorectal cancers, prostate cancer, kidney cancer, melanomas, mantel cell lymphoma (MCL), chronic myeloid leukemia (CML), acute myeloid leukemia (AML), or the like.

### BRIEF DESCRIPTION OF THE DRAWINGS

FIG. 1 shows the efficacy of a Abemaciclib/TMZ combination. Dosing: TMZ, QD×5; 6 mg/kg+abemaciclib, PO, QD×21, 100 mg/kg.

FIG. 2 shows the efficacy of a Compound 1/TMZ combination. Dosing: TMZ: QD×5; 6 mg/kg+Compound 1, PO, QD×21, 100 mg/kg.

### DETAILED DESCRIPTION OF THE INVENTION

One aspect of the invention is directed to a method of treating a brain cancer or brain metastases originated from other cancers, comprising administering to a subject in need thereof, a therapeutically effective amount of a composition comprising a compound of formula (I):

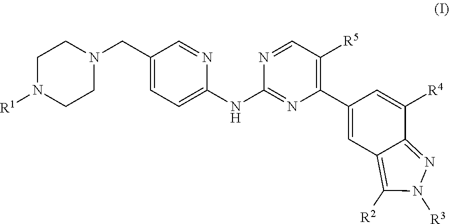

or a pharmaceutically acceptable salt, solvate, or prodrug thereof, wherein:

R¹ is hydrogen, C₁-C₈ alkyl, C₃-C₇ cycloalkyl, R⁶C(O)—, or R⁷O(CO)—;

R² and R³ are each independently hydrogen, C₁-C₈ alkyl, C₃-C₇ cycloalkyl, or C₃-C₇ cycloalkylmethyl;

R⁴ is hydrogen, halogen, C₁-C₈ alkyl, or C₃-C₇ cycloalkyl;

R⁵ is hydrogen or halogen. R¹ can be C₁-C₆ alkyl;

R⁶ is hydrogen, C₁-C₈ alkyl; or C₃-C₇ cycloalkyl; and

R⁷ is C₁-C₈ alkyl; or C₃-C₇ cycloalkyl,

wherein any said alkyl or cycloalkyl is optionally substituted.

In one embodiment, R¹ is hydrogen, methyl, ethyl, propyl, or isopropyl.

In another embodiment, R² can be C₁-C₆ alkyl, C₃-C₆ cycloalkyl, or C₃-C₆ cycloalkylmethyl.

In another embodiment, R² is methyl, ethyl, propyl, isopropyl, cyclopropyl, cyclopentyl, cyclopropylmethyl, or cyclopentylmethyl.

In another embodiment, R³ can be C₁-C₆ alkyl or C₃-C₆ cycloalkyl.

In another embodiment, R³ is methyl, ethyl, propyl, isopropyl, or cyclopropyl.

In another embodiment, R⁴ is hydrogen or halogen.

In another embodiment, R⁵ is hydrogen or fluoro.

In another embodiment, sometimes preferably, R¹ is methyl or ethyl; R² is isopropyl, cyclopropyl, cyclopropylmethyl, or cyclopentyl; R³ is methyl or ethyl; R⁴ is hydrogen or fluoro; and R⁵ is hydrogen or fluoro.

In another embodiment, the invention encompasses any combination of the embodiments described herein.

Preferably, the brain cancer or the metastatic cancer being treated expresses CDK4 and/or CDK6. Preferably, the brain cancer is a glioblastoma.

Another aspect of the invention is directed to a method of treating a brain cancer or brain metastases originated from other cancers, comprising administering to a subject in need thereof, a therapeutically effective amount of a composition comprising a compound of formula:

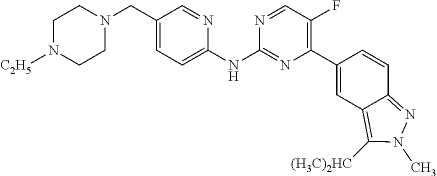

or a pharmaceutically acceptable salt, solvate, or prodrug thereof. Preferably, the brain cancer or the metastatic cancer being treated expresses CDK4 and/or CDK6. Preferably, the brain cancer is a glioblastoma.

A further aspect of the invention is directed to use of a compound of formula (I):

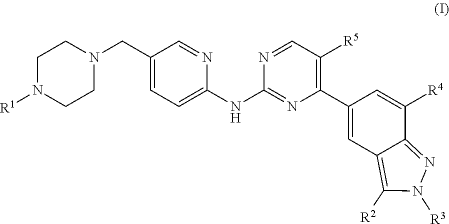

or a pharmaceutically acceptable salt, solvate, or prodrug thereof, in the manufacture of a medicament for treatment of a brain cancer associated with CDK4 and/or CDK6 activity, wherein:

R¹ is hydrogen, C₁-C₈ alkyl, or C₃-C₇ cycloalkyl;

R² and R³ are each independently hydrogen, C₁-C₈ alkyl, C₃-C₇ cycloalkyl, or C₃-C₇ cycloalkylmethyl;

R⁴ is hydrogen, halogen, C₁-C₈ alkyl, or C₃-C₇ cycloalkyl; and

R⁵ is hydrogen or halogen.

In some embodiments, R¹ is C₁-C₆ alkyl. Preferably, R¹ is methyl, ethyl, propyl, or isopropyl.

In some embodiments, R² is C₁-C₆ alkyl, C₃-C₆ cycloalkyl, or C₃-C₆ cycloalkylmethyl. Preferably, R² is methyl, ethyl, propyl, isopropyl, cyclopropyl, cyclopentyl, cyclopropylmethyl, or cyclopentylmethyl.

In some embodiments, R³ is C₁-C₆ alkyl or C₃-C₆ cycloalkyl. Preferably, R³ is methyl, ethyl, propyl, isopropyl, or cyclopropyl.

In some embodiments, R⁴ is hydrogen or halogen.

In some embodiments, R⁵ is hydrogen or fluoro.

In some embodiments, sometimes more preferably, R¹ is methyl or ethyl; R² is isopropyl, cyclopropyl, cyclopropylmethyl, or cyclopentyl; R³ is methyl or ethyl; R is hydrogen or fluoro; and R⁵ is hydrogen or fluoro.

In some preferred embodiments, sometimes preferably, the brain cancer associated with CDK4 and/or CDK6 activity is a glioblastoma or brain metastasis of another cancer.

Another aspect of the invention is directed to use of a compound of the formula:

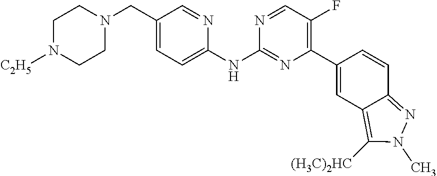

or a pharmaceutically acceptable salt, solvate, or prodrug thereof, in the manufacture of a medicament for the treatment of a brain cancer or metastatic cancer associated with CDK4 and/or CDK6 activity, such as a metastatic brain cancer. Preferably, the brain cancer is a glioblastoma.

In any of the embodiments described above, the cancers that are associated with CDK4 and/or CDK6 activity and cause brain metastasis include, but are not limited to, breast cancers, lung cancers (especially non-small cell lung cancer (NSCLC)), colorectal cancers, prostate cancer, kidney cancer, melanomas, mantel cell lymphoma (MCL), chronic myeloid leukemia (CML), acute myeloid leukemia (AML), or the like, the method comprising administering to a cancer patient with a therapeutically effective amount of the compound according to any embodiment disclosed herein.

In a preferred embodiment, the method is directed to treatment of metastatic breast cancer.

In another preferred embodiment, the method is directed to treatment of metastatic lung cancer, in particular, metastatic non-small cell lung cancer.

In some embodiments, the present invention provides a method of using the compounds disclosed herein on a cancer patient for a prophylactic effect in preventing the brain metastasis, i.e., spread of cancer cells from the original diseased organs.

In all the embodiments, preferably, the brain cancer or brain metastases are associated the activity of CDK, in particular, CDK4 or CDK6, activity.

The present invention encompasses all possible combinations of any embodiments disclosed herein.

Unless otherwise indicated, the term “alkyl,” as used herein, is intended to include both branched and straight-chain saturated aliphatic hydrocarbon groups containing 1 to 8 carbons, preferably 1 to 6, more preferably 1 to 4, carbons. The term encompasses, but is not limited to, methyl, ethyl, propyl, isopropyl, butyl, t-butyl, isobutyl, pentyl, hexyl, or the like.

Unless otherwise indicated, the term “alkylene,” as used herein, refers to a bivalent saturated aliphatic radical derived from an alkane by removal of two hydrogen atoms. Examples include, but are not limited to, methylene (—CH₂—), ethylene (—CH₂CH₂—), propylene (—CH₂CH₂CH₂—), or the like.

Unless otherwise indicated, the term “cycloalkyl”, as used herein alone or as a part of another group, includes saturated cyclic hydrocarbon radical having 3 to 8, sometimes preferably 3-6, carbons forming the ring. Examples include, but are not limited to, cyclopropyl, cyclobutyl, cyclopentyl, and cyclohexyl.

“Halo” or “halogen” as used herein, refers to fluoro (F), chloro (Cl), bromo (Br), and iodo (I).

Further, in any embodiment disclosed herein, the alkyl, alkylene, cycloalkyl, and cycloalkylmethyl groups may each optionally be independently substituted by one or more, preferably one to three, sometimes preferably one to two, substituent(s) independently selected from the group consisting of halogen, C₁-C₄ alkyl, OH, C₁-C₄ alkoxy, and CN.

When any group is said to be “optionally substituted,” unless specifically defined, it means that the group is or is not substituted, provided that such substitution would not violate the conventional bonding principles known to a person of ordinary skill in the art. When the phrase “optionally substituted” is used before a list of groups, it means that each one of the groups listed may be optionally substituted.

One of ordinary skill in the art would understand that with respect to any molecule described as containing one or more substituents, only sterically practical and/or synthetically feasible compounds are meant to be included. Unless otherwise specified in this specification, when a variable is said to optionally substituted or substituted with a substituent(s), this is to be understood that this substitution occurs by replacing a hydrogen that is covalently bound to the variable with one of these substituent(s).

The compounds of the present invention are generally recognized as organic bases, which are able to react with acids, specifically pharmaceutically acceptable acids, to form pharmaceutically acceptable salts.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. See, e.g., S. M. Berge et al., J. Pharm. Sci., 1977, 66, 1-19, which is incorporated herein by reference. Pharmaceutically acceptable salts of the compounds of this invention include those derived from suitable inorganic and organic acids. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Preferred pharmaceutically acceptable salts include the hydrochloride salts.

The term “solvate,” as used herein, means a physical association of a compound of this invention with a stoichiometric or non-stoichiometric amount of solvent molecules. For example, one molecule of the compound associates with one or more, preferably one to three, solvent molecules. It is also possible that multiple (e.g., 1.5 or 2) molecules of the compound share one solvent molecule. This physical association may include hydrogen bonding. In certain instances the solvates will be capable of isolation as crystalline solid. The solvent molecules in the solvate may be present in a regular arrangement and/or a non-ordered arrangement. Exemplary solvates include, but are not limited to, hydrates, ethanolates, methanolates, and isopropanolates. Methods of solvation are generally known in the art.

Although the compounds of general formula (I) disclosed herein may be in the “prodrug” forms themselves, i.e., when R¹ is an acyl (i.e., RC(O)—) or ester (i.e., ROC(O)—) group, these “prodrugs” may be generated in vivo under physiological conditions from other “prodrugs”. Thus, for these compounds disclosed, the term “prodrug,” as used herein, refers to a derivative of a compound that can be transformed in vivo to yield the parent compound, for example, by hydrolysis in blood. Common examples of prodrugs in the present invention include, but are not limited to, amide or phosphoramide forms of an active amine compound, for example, the compound of formula (II):

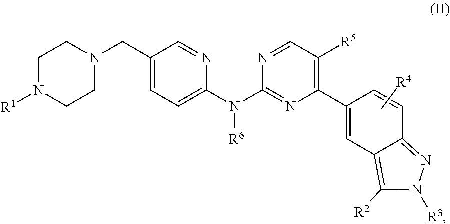

wherein R⁶ is an acyl group (e.g., acetyl, propionyl, formyl, etc.) or phosphoryl [e.g., —P(═O)(OH)₂] group; or alternatively, when R³ in an active compound is hydrogen, the corresponding amide or phosphoramide compounds may serve as prodrugs. Such amide or phosphoramide prodrug compounds may be prepared according to conventional methods as known in the art.

While it is possible that, for use in therapy, therapeutically effective amounts of a compound of the present invention, or pharmaceutically acceptable salts or solvates thereof, may be administered as the raw chemical, it is possible to present the active ingredient as a pharmaceutical composition. Accordingly, the disclosure further provides pharmaceutical compositions, which include any compounds of the present invention, or pharmaceutically acceptable salts or solvates thereof, and one or more, preferably one to three, pharmaceutically acceptable carriers, diluents, or other excipients. The carrier(s), diluent(s), or other excipient(s) must be acceptable in the sense of being compatible with the other ingredients of the formulation and not deleterious to the subject being treated.

The term “pharmaceutically acceptable,” as used herein, refers to the property of those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

Pharmaceutical formulations may be presented in unit dose forms containing a predetermined amount of active ingredient per unit dose. Typically, the pharmaceutical compositions of this disclosure will be administered from once every 1 to 5 days to about 1-5 times per day, or alternatively, as a continuous infusion. Such administration can be used as a chronic or acute therapy. The amount of active ingredient that may be combined with the carrier materials to produce a single dosage form will vary depending on the condition being treated, the severity of the condition, the time of administration, the route of administration, the rate of excretion of the compound employed, the duration of treatment, and the age, gender, weight, and condition of the patient. Preferred unit dosage formulations are those containing a daily dose or sub-dose, as herein above recited, or an appropriate fraction thereof, of an active ingredient. Generally, treatment is initiated with small dosages substantially less than the optimum dose of the compound. Thereafter, the dosage is increased by small increments until the optimum effect under the circumstances is reached. In general, the compound is most desirably administered at a concentration level that will generally afford effective results without causing substantial harmful or deleterious side effects.

When the compositions of this disclosure comprise a combination of a compound of the present disclosure and one or more, preferably one or two, additional therapeutic or prophylactic agent, both the compound and the additional agent are usually present at dosage levels of between about 10 to 150%, and more preferably between about 10 and 80% of the dosage normally administered in a monotherapy regimen.

Pharmaceutical formulations may be adapted for administration by any appropriate route, for example, by the oral (including buccal or sublingual), rectal, nasal, topical (including buccal, sublingual, or transdermal), vaginal, or parenteral (including subcutaneous, intracutaneous, intramuscular, intra-articular, intrasynovial, intrasternal, intrathecal, intralesional, intravenous, or intradermal injections or infusions) route. Such formulations may be prepared by any method known in the art of pharmacy, for example by bringing into association the active ingredient with the carrier(s) or excipient(s). Oral administration or administration by injection are preferred.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil emulsions.

For instance, for oral administration in the form of a tablet or capsule, the active drug component can be combined with an oral, non-toxic pharmaceutically acceptable inert carrier such as ethanol, glycerol, water, and the like. Powders are prepared by comminuting the compound to a suitable fine size and mixing with a similarly comminuted pharmaceutical carrier such as an edible carbohydrate, as, for example, starch or mannitol. Flavoring, preservative, dispersing, and coloring agent can also be present.

Capsules are made by preparing a powder mixture, as described above, and filling formed gelatin sheaths. Glidants and lubricants such as colloidal silica, talc, magnesium stearate, calcium stearate, or solid polyethylene glycol can be added to the powder mixture before the filling operation. A disintegrating or solubilizing agent such as agar-agar, calcium carbonate, or sodium carbonate can also be added to improve the availability of the medicament when the capsule is ingested.

Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, and the like. Lubricants used in these dosage forms include sodium oleate, sodium chloride, and the like. Disintegrators include, without limitation, starch, methyl cellulose, agar, betonite, xanthan gum, and the like. Tablets are formulated, for example, by preparing a powder mixture, granulating or slugging, adding a lubricant and disintegrant, and pressing into tablets. A powder mixture is prepared by mixing the compound, suitable comminuted, with a diluent or base as described above, and optionally, with a binder such as carboxymethylcellulose, an aliginate, gelating, or polyvinyl pyrrolidone, a solution retardant such as paraffin, a resorption accelerator such as a quaternary salt and/or and absorption agent such as betonite, kaolin, or dicalcium phosphate. The powder mixture can be granulated by wetting with a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compounds of the present disclosure can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solution, syrups, and elixirs can be prepared in dosage unit form so that a given quantity contains a predetermined amount of the compound. Syrups can be prepared by dissolving the compound in a suitably flavored aqueous solution, while elixirs are prepared through the use of a non-toxic vehicle. Solubilizers and emulsifiers such as ethoxylated isostearyl alcohols and polyoxyethylene sorbitol ethers, preservatives, flavor additive such as peppermint oil or natural sweeteners, or saccharin or other artificial sweeteners, and the like can also be added.

Where appropriate, dosage unit formulations for oral administration can be microencapsulated. The formulation can also be prepared to prolong or sustain the release, for example, by coating or embedding particulate material in polymers, wax, or the like.

It should be understood that in addition to the ingredients particularly mentioned above, the formulations may include other agents conventional in the art having regard to the type of formulation in question, for example those suitable for oral administration may include flavoring agents.

The term “subject” or “patient” includes both humans and other mammalian animals, including but not limited horses, dogs, cats, pigs, monkeys, etc., preferably humans.

The term “therapeutically effective amount” refers to an amount of a compound or composition that, when administered to a subject for treating a disease, is sufficient to effect such treatment for the disease. A “therapeutically effective amount” can vary depending on, inter alia, the compound, the disease and its severity, and the age, weight, or other factors of the subject to be treated. When applied to an individual active ingredient, administered alone, the term refers to that ingredient alone. When applied to a combination, the term refers to combined amounts of the active ingredients that result in the therapeutic effect, whether administered in combination, serially, or simultaneously.

In some embodiments, the term “treating” or “treatment” refers to: (i) inhibiting the disease, disorder, or condition, i.e., arresting its development; (ii) relieving the disease, disorder, or condition, i.e., causing regression of the disease, disorder, and/or condition; or (iii) preventing a disease, disorder or condition from occurring in a subject that may be predisposed to the disease, disorder, and/or condition but has not yet been diagnosed as having it. Thus, in some embodiments, “treating” or “treatment” refers to ameliorating a disease or disorder, which may include ameliorating one or more physical parameters, though maybe indiscernible by the subject being treated. In some embodiments, “treating” or “treatment” includes modulating the disease or disorder, either physically (e.g., stabilization of a discernible symptom) or physiologically (e.g., stabilization of a physical parameter) or both. In yet some embodiments, “treating” or “treatment” includes delaying the onset of the disease or disorder.

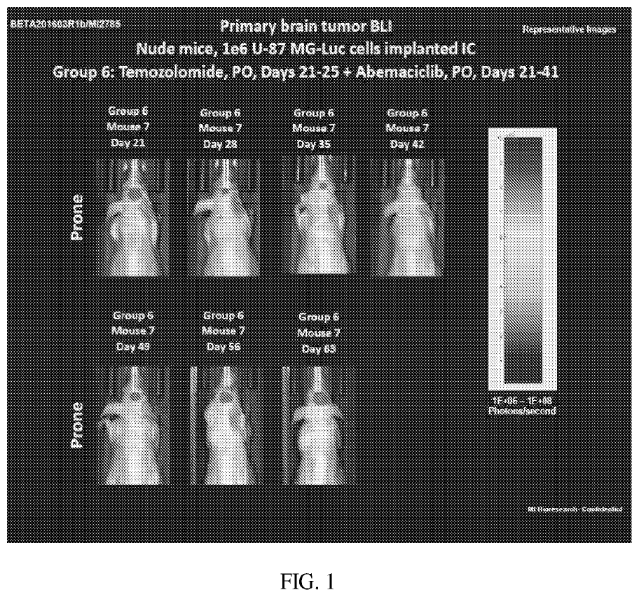

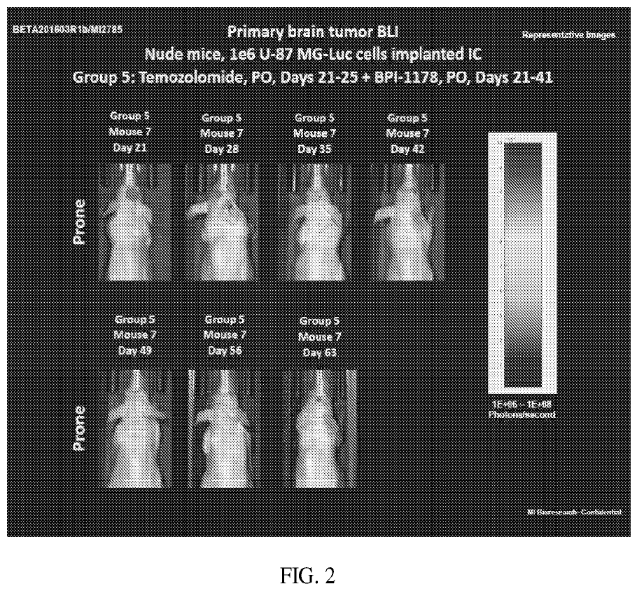

An efficacy and comparison study between Compound 1 and abemaciclib, in combination with temozolomide (TMZ), against orthotopic U87MG-luc human glioblastoma in mice was conducted. In each study, TMZ was dosed PO at 6 mg/kg, QD×5, and either Compound 1 or abemaciclib was dosed PO at 100 mg/kg. Tumor growth was observed by bioluminescence. The abemaciclib/TMZ combination showed tumor volume reduction up to day 42, followed by regrowth at day 49 (FIG. 1). In contrast, the Compound 1/TMZ combination showed significant tumor volume reduction at day 28, with sustained tumor volume reduction through day 63 (FIG. 2). Given that the in vitro potencies of Compound 1 and abemaciclib are comparable, the superior in vivo efficacy of Compound 1 relative to abemaciclib in a glioblastoma model can be attributed to the more favorable BBB permeability profile of Compound 1 vs. abemaciclib. From a broad perspective, the significant differentiation between Compound 1 and abemaciclib in a brain disease model can be traced to their distinct molecular structures.

The major difference in molecular structure between Compound 1 and abemaciclib is that Compound 1 contains a 2H-indazole nucleus, whereas abemaciclib contains a benzimidazole nucleus:

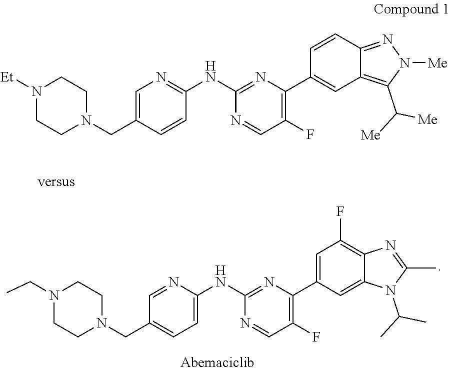 [SMILES: CCN1CCN(Cc2ccc(Nc3ncc(F)c(-c4ccc5nn(C)c(C(C)C)c5c4)n3)nc2)CC1 ; CCN1CCN(Cc2ccc(Nc3ncc(F)c(-c4cc(F)c5nc(C)n(C(C)C)c5c4)n3)nc2)CC1]

This structural differentiation surprisingly results in a significant BBB permeability profile difference between the two compounds. Example 3 describes in vivo mouse studies, where the brain concentration of Compound 1 was observed to be approximately 3-fold higher than that of abemaciclib, and the brain/plasma (B/P) ratio for Compound 1 was 1.43 vs. 0.43 for abemaciclib (see Tables 1 and 2). Further, and notably, Compound 1 is not a P-gp substrate (see Example 2).

TABLE 1
Brain concentrations and B/P ratios of Compound 1 in mouse at
10 mg/kg p.o.

| | Individual and Mean Concentration of Compound 1 | | | | | | |
|---|---|---|---|---|---|---|---|
| | in Mouse after PO Administration at 10 mg/kg | | | | | | |
| | Plasma Concentration of Compound 1 (ng/mL) | | | | | | |
| | | | | | | | CV |
| | Time (h) | R1 + 3n | R2 + 3n | R3 + 3n | Mean PO | SD | (%) |
| n = | 2.00 | 833 | 500 | 748 | 694 | 173 | 24.9 |
| 0 | | | | | | | |
| n = | 4.00 | 669 | 543 | 1180 | 797 | 337 | 42.3 |
| 1 | | | | | | | |
| n = | 8.00 | 868 | 1030 | 722 | 873 | 154 | 17.6 |
| 2 | | | | | | | |
| n = | 24.0 | 4.36 | 5.30 | 10.6 | 6.75 | 3.36 | 49.8 |
| 3 | | | | | | | |
| | AUC₀₋ₗₐₛₜ | | | | 8375 | | |
| | (ng · | | | | | | |
| | h/mL) | | | | | | |
| | ^{a}Brain Concentration of Compound 1 (ng/g) | | | | | | |
| | | | | | | | CV |
| | Time (h) | R1 + 3n | R2 + 3n | R3 + 3n | Mean PO | SD | (%) |
| n = | 2.00 | 954 | 630 | 1098 | 894 | 240 | 26.8 |
| 0 | | | | | | | |
| n = | 4.00 | 1194 | 1062 | 1218 | 1158 | 84.0 | 7.25 |
| 1 | | | | | | | |
| n = | 8.00 | 1152 | 1380 | 1218 | 1250 | 117 | 9.39 |
| 2 | | | | | | | |
| n = | 24.0 | 9.06 | 8.64 | 15.8 | 11.2 | 4.04 | 36.1 |
| 3 | | | | | | | |
| | AUC₀₋ₗₐₛₜ | | | | 11966 | | |
| | (ng · h/g) | | | | | | |
| | ^{d}AUC₀₋ₗₐₛₜ | | | | 1.43 | | |
| | Ratio | | | | | | |

TABLE 2
Brain concentrations and B/P ratios of abemaciclib in mouse at
10 mg/kg p.o.

| | Individual and Mean Concentration of abemaciclib (2) in | | | | | | |
|---|---|---|---|---|---|---|---|
| | Mouse after PO at 10 mg/kg | | | | | | |
| | Plasma Concentration of abemaciclib (ng/mL) | | | | | | |
| | | | | | | | CV |
| | Time (h) | R1 + 3n | R2 + 3n | R3 + 3n | Mean PO | SD | (%) |
| n = | 2.00 | 633 | 1055 | 821 | 836 | 211 | 25.3 |
| 0 | | | | | | | |
| n = | 4.00 | 700 | 744 | 963 | 802 | 141 | 17.5 |
| 1 | | | | | | | |
| n = | 8.00 | 1025 | 707 | 780 | 837 | 167 | 19.9 |
| 2 | | | | | | | |
| n = | 24.0 | 11.5 | 46.7 | 16.6 | 24.9 | 19.1 | 76.5 |
| 3 | | | | | | | |
| | AUC₀₋ₗₐₛₜ | | | | 9449 | | |
| | (ng · | | | | | | |
| | h/mL) | | | | | | |
| | ^{a}Brain Concentration of abemaciclib (ng/g) | | | | | | |
| | | | | | | | CV |
| | Time (h) | R1 + 3n | R2 + 3n | R3 + 3n | Mean PO | SD | (%) |
| n = | 2.00 | 216 | 326 | 302 | 282 | 57.7 | 20.5 |
| 0 | | | | | | | |
| n = | 4.00 | 452 | 370 | 469 | 430 | 52.7 | 12.2 |
| 1 | | | | | | | |
| n = | 8.00 | 421 | 277 | 341 | 347 | 72.2 | 20.8 |
| 2 | | | | | | | |
| n = | 24.0 | 6.51 | 14.8 | 10.6 | 10.6 | 4.16 | 39.1 |
| 3 | | | | | | | |
| | AUC₀₋ₗₐₛₜ | | | | 4085 | | |
| | (ng · h/g) | | | | | | |
| | ^{d}AUC₀₋ₗₐₛₜ | | | | 0.432 | | |
| | Ratio | | | | | | |

While not intending to be limited, illustrated non-limiting examples of the compounds that can be used for the present invention are listed in Table 3.

TABLE 3
Selected examples of the compounds of formula (I)

| Example | Structure | Name |
|---|---|---|
| 1 | 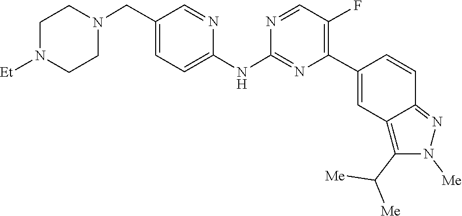 [SMILES: CCN1CCN(Cc2ccc(Nc3ncc(F)c(-c4ccc5nn(C)c(C(C)C)c5c4)n3)nc2)CC1] | N-(5-((4-ethylpiperazin-1- yl)methyl)pyridin-2-yl)-5- fluoro-4-(3-isopropyl-2- methyl-2H-indazol-5- yl)pyrimidin-2-amine |
| 2 | 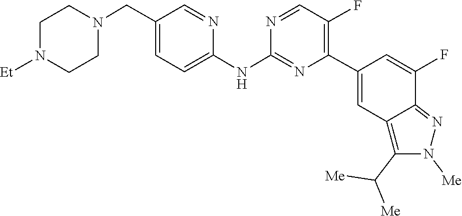 [SMILES: CCN1CCN(Cc2ccc(Nc3ncc(F)c(-c4cc(F)c5nn(C)c(C(C)C)c5c4)n3)nc2)CC1] | N-(5-((4-ethylpiperazin-1- yl)methyl)pyridin-2-yl)-5- fluoro-4-(7-fluoro-3- isopropyl-2-methyl-2H- indazol-5-yl)pyrimidin-2- amine |
| 3 | 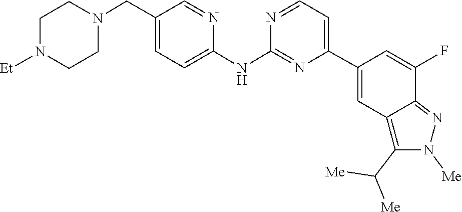 [SMILES: CCN1CCN(Cc2ccc(Nc3nccc(-c4cc(F)c5nn(C)c(C(C)C)c5c4)n3)nc2)CC1] | N-(5-((4-ethylpiperazin-1- yl)methyl)pyridin-2-yl)-4-(7- fluoro-3-isopropyl-2-methyl- 2H-indazol-5-yl)pyrimidin-2- amine |
| 4 | 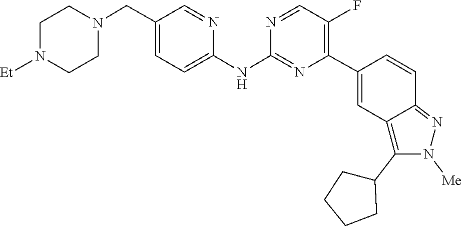 [SMILES: CCN1CCN(Cc2ccc(Nc3ncc(F)c(-c4ccc5nn(C)c(C6CCCC6)c5c4)n3)nc2)CC1] | 4-(3-cyclopentyl-2-methyl- 2H-indazol-5-yl)-N-(5-((4- ethylpiperazin-1- yl)methyl)pyridin-2-yl)-5- fluoropyrimidin-2-amine |
| 5 | 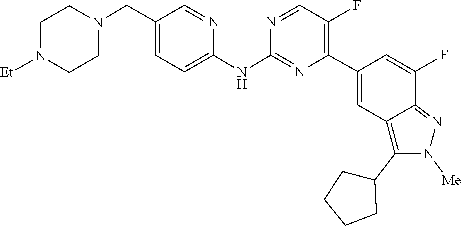 [SMILES: CCN1CCN(Cc2ccc(Nc3ncc(F)c(-c4cc(F)c5nn(C)c(C6CCCC6)c5c4)n3)nc2)CC1] | 4-(3-cyclopentyl-7-fluoro-2- methyl-2H-indazol-5-yl)-N- (5-((4-ethylpiperazin-1- yl)methyl)pyridin-2-yl)-5- fluoropyrimidin-2-amine |
| 6 | 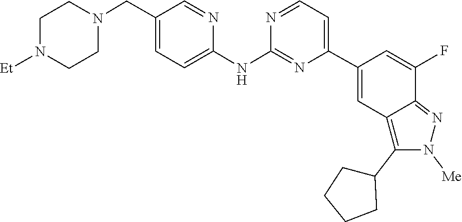 [SMILES: CCN1CCN(Cc2ccc(Nc3nccc(-c4cc(F)c5nn(C)c(C6CCCC6)c5c4)n3)nc2)CC1] | 4-(3-cyclopentyl-7-fluoro-2- methyl-2H-indazol-5-yl)-N- (5-((4-ethylpiperazin-1- yl)methyl)pyridin-2- yl)pyrimidin-2-amine |
| 7 | 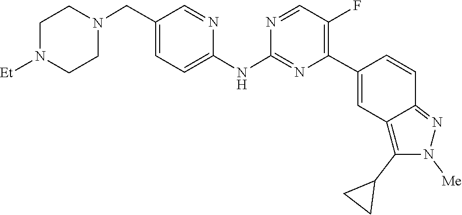 [SMILES: CCN1CCN(Cc2ccc(Nc3ncc(F)c(-c4ccc5nn(C)c(C6CC6)c5c4)n3)nc2)CC1] | 4-(3-cyclopropyl-2-methyl- 2H-indazol-5-yl)-N-(5-((4- ethylpiperazin-1- yl)methyl)pyridin-2-yl)-5- fluoropyrimidin-2-amine |
| 8 | 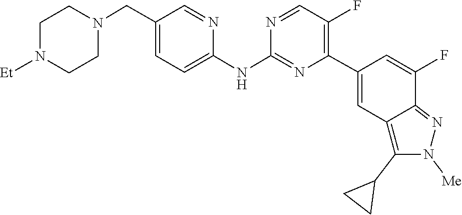 [SMILES: CCN1CCN(Cc2ccc(Nc3ncc(F)c(-c4cc(F)c5nn(C)c(C6CC6)c5c4)n3)nc2)CC1] | 4-(3-cyclopropyl-7-fluoro-2- methyl-2H-indazol-5-yl)-N- (5-((4-ethylpiperazin-1- yl)methyl)pyridin-2-yl)-5- fluoropyrimidin-2-amine |
| 9 | 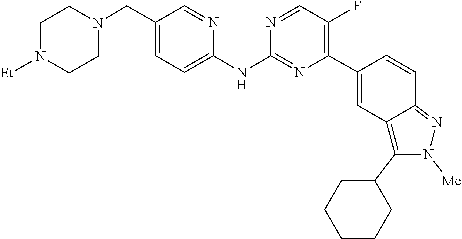 [SMILES: CCN1CCN(Cc2ccc(Nc3ncc(F)c(-c4ccc5nn(C)c(C6CCCCC6)c5c4)n3)nc2)CC1] | 4-(3-cyclohexyl-2-methyl- 2H-indazol-5-yl)-N-(5-((4- ethylpiperazin-1- yl)methyl)pyridin-2-yl)-5- fluoropyrimidin-2-amine |
| 10 | 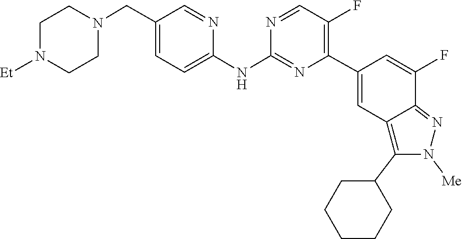 [SMILES: CCN1CCN(Cc2ccc(Nc3ncc(F)c(-c4cc(F)c5nn(C)c(C6CCCCC6)c5c4)n3)nc2)CC1] | 4-(3-cyclohexyl-7-fluoro-2- methyl-2H-indazol-5-yl)-N- (5-((4-ethylpiperazin-1- yl)methyl)pyridin-2-yl)-5- fluoropyrimidin-2-amine |
| 11 | 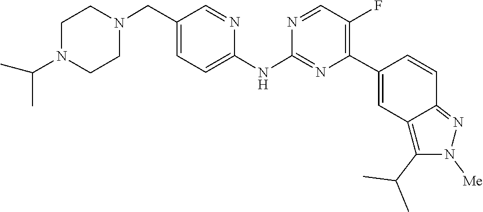 [SMILES: CC(C)c1c2cc(-c3nc(Nc4ccc(CN5CCN(C(C)C)CC5)cn4)ncc3F)ccc2nn1C] | 5-fluoro-4-(3-isopropyl-2- methyl-2H-indazol-5-yl)-N- (5-((4-isopropylpiperazin-1- yl)methyl)pyridin-2- yl)pyrimidin-2-amine |
| 12 | 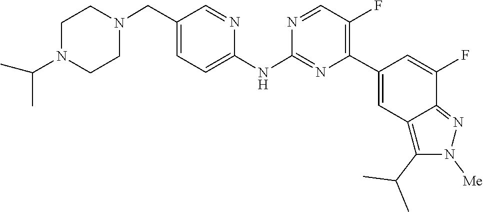 [SMILES: CC(C)c1c2cc(-c3nc(Nc4ccc(CN5CCN(C(C)C)CC5)cn4)ncc3F)cc(F)c2nn1C] | 5-fluoro-4-(7-fluoro-3- isopropyl-2-methyl-2H- indazol-5-yl)-N-(5-((4- isopropylpiperazin-1- yl)methyl)pyridin-2- yl)pyrimidin-2-amine |
| 13 | 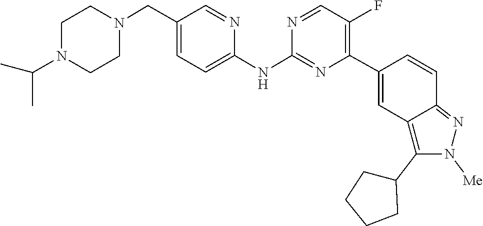 [SMILES: CC(C)N1CCN(Cc2ccc(Nc3ncc(F)c(-c4ccc5nn(C)c(C6CCCC6)c5c4)n3)nc2)CC1] | 4-(3-cyclopentyl-2-methyl- 2H-indazol-5-yl)-5-fluoro-N- (5-((4-isopropylpiperazin-1- yl)methyl)pyridin-2- yl)pyrimidin-2-amine |
| 14 | 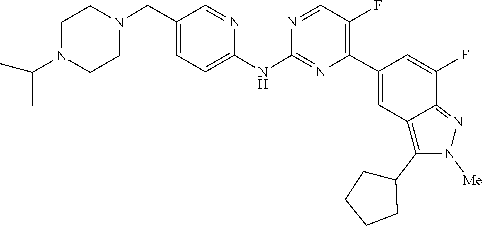 [SMILES: CC(C)N1CCN(Cc2ccc(Nc3ncc(F)c(-c4cc(F)c5nn(C)c(C6CCCC6)c5c4)n3)nc2)CC1] | 4-(3-cyclopentyl-7-fluoro-2- methyl-2H-indazol-5-yl)-5- fluoro-N-(5-((4- isopropylpiperazin-1- yl)methyl)pyridin-2- yl)pyrimidin-2-amine |
| 15 | 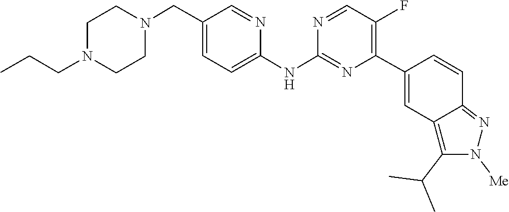 [SMILES: CCCN1CCN(Cc2ccc(Nc3ncc(F)c(-c4ccc5nn(C)c(C(C)C)c5c4)n3)nc2)CC1] | 5-fluoro-4-(3-isopropyl-2- methyl-2H-indazol-5-yl)-N- (5-((4-propylpiperazin-1- yl)methyl)pyridin-2- yl)pyrimidin-2-amine |
| 16 | 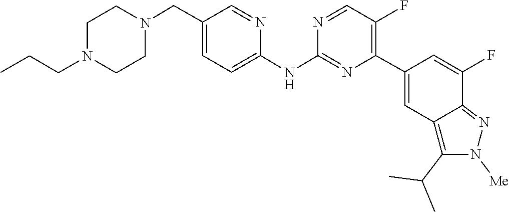 [SMILES: CCCN1CCN(Cc2ccc(Nc3ncc(F)c(-c4cc(F)c5nn(C)c(C(C)C)c5c4)n3)nc2)CC1] | 5-fluoro-4-(7-fluoro-3- isopropyl-2-methyl-2H- indazol-5-yl)-N-(5-((4- propylpiperazin-1- yl)methyl)pyridin-2- yl)pyrimidin-2-amine |
| 17 | 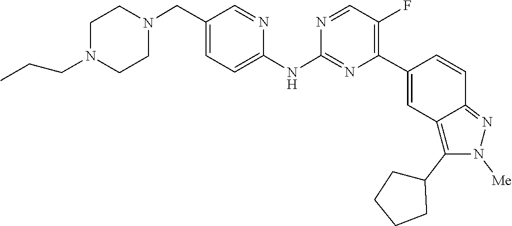 [SMILES: CCCN1CCN(Cc2ccc(Nc3ncc(F)c(-c4ccc5nn(C)c(C6CCCC6)c5c4)n3)nc2)CC1] | 4-(3-cyclopentyl-2-methyl- 2H-indazol-5-yl)-5-fluoro-N- (5-((4-propylpiperazin-1- yl)methyl)pyridin-2- yl)pyrimidin-2-amine |
| 18 | 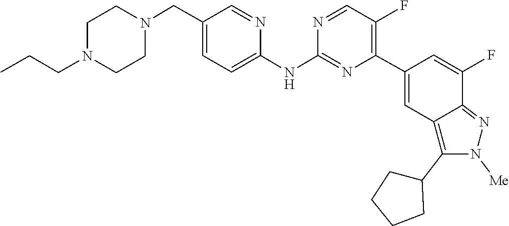 [SMILES: CCCN1CCN(Cc2ccc(Nc3ncc(F)c(-c4cc(F)c5nn(C)c(C6CCCC6)c5c4)n3)nc2)CC1] | 4-(3-cyclopentyl-7-fluoro-2- methyl-2H-indazol-5-yl)-5- fluoro-N-(5-((4- propylpiperazin-1- yl)methyl)pyridin-2- yl)pyrimidin-2-amine |
| 19 | 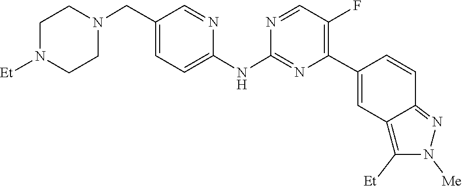 [SMILES: CCc1c2cc(-c3nc(Nc4ccc(CN5CCN(CC)CC5)cn4)ncc3F)ccc2nn1C] | 4-(3-ethyl-2-methyl-2H- indazol-5-yl)-N-(5-((4- ethylpiperazin-1- yl)methyl)pyridin-2-yl)-5- fluoropyrimidin-2-amine |
| 20 | 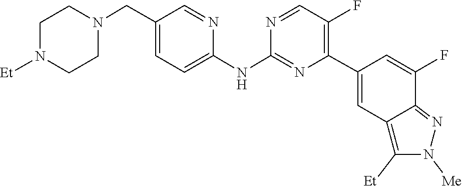 [SMILES: CCc1c2cc(-c3nc(Nc4ccc(CN5CCN(CC)CC5)cn4)ncc3F)cc(F)c2nn1C] | 4-(3-ethyl-7-fluoro-2-methyl- 2H-indazol-5-yl)-N-(5-((4- ethylpiperazin-1- yl)methyl)pyridin-2-yl)-5- fluoropyrimidin-2-amine |
| 21 | 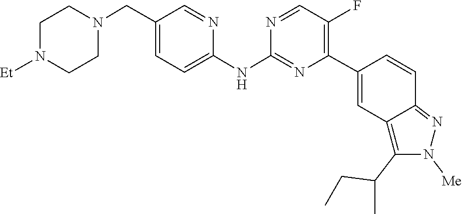 [SMILES: CCC(C)c1c2cc(-c3nc(Nc4ccc(CN5CCN(CC)CC5)cn4)ncc3F)ccc2nn1C] | 4-(3-(sec-butyl)-2-methyl- 2H-indazol-5-yl)-N-(5-((4- ethylpiperazin-1- yl)methyl)pyridin-2-yl)-5- fluoropyrimidin-2-amine |
| 22 | 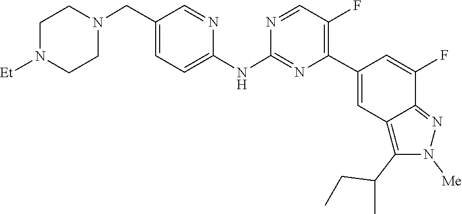 [SMILES: CCC(C)c1c2cc(-c3nc(Nc4ccc(CN5CCN(CC)CC5)cn4)ncc3F)cc(F)c2nn1C] | 4-(3-(sec-butyl)-7-fluoro-2- methyl-2H-indazol-5-yl)-N- (5-((4-ethylpiperazin-1- yl)methyl)pyridin-2-yl)-5- fluoropyrimidin-2-amine |
| 23 | 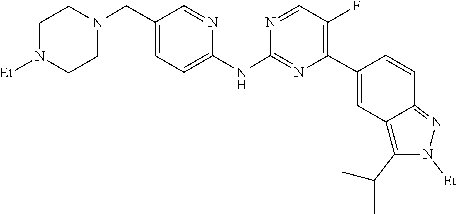 [SMILES: CCN1CCN(Cc2ccc(Nc3ncc(F)c(-c4ccc5nn(CC)c(C(C)C)c5c4)n3)nc2)CC1] | 4-(2-ethyl-3-isopropyl-2H- indazol-5-yl)-N-(5-((4- ethylpiperazin-1- yl)methyl)pyridin-2-yl)-5- fluoropyrimidin-2-amine |
| 24 | 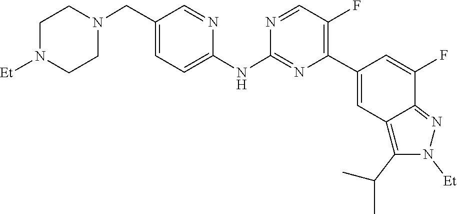 [SMILES: CCN1CCN(Cc2ccc(Nc3ncc(F)c(-c4cc(F)c5nn(CC)c(C(C)C)c5c4)n3)nc2)CC1] | 4-(2-ethyl-7-fluoro-3- isopropyl-2H-indazol-5-yl)- N-(5-((4-ethylpiperazin-1- yl)methyl)pyridin-2-yl)-5- fluoropyrimidin-2-amine |
| 25 | 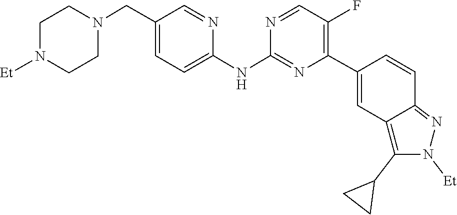 [SMILES: CCN1CCN(Cc2ccc(Nc3ncc(F)c(-c4ccc5nn(CC)c(C6CC6)c5c4)n3)nc2)CC1] | 4-(3-cyclopropyl-2-ethyl-2H- indazol-5-yl)-N-(5-((4- ethylpiperazin-1- yl)methyl)pyridin-2-yl)-5- fluoropyrimidin-2-amine |
| 26 | 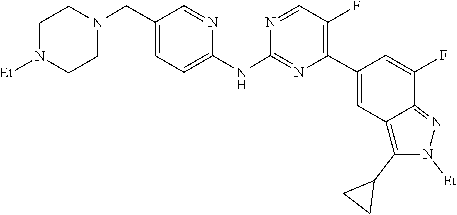 [SMILES: CCN1CCN(Cc2ccc(Nc3ncc(F)c(-c4cc(F)c5nn(CC)c(C6CC6)c5c4)n3)nc2)CC1] | 4-(3-cyclopropyl-2-ethyl-7- fluoro-2H-indazol-5-yl)-N-(5- ((4-ethylpiperazin-1- yl)methyl)pyridin-2-yl)-5- fluoropyrimidin-2-amine |
| 27 | 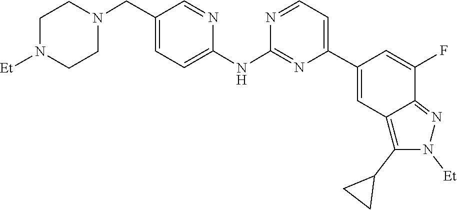 [SMILES: CCN1CCN(Cc2ccc(Nc3nccc(-c4cc(F)c5nn(CC)c(C6CC6)c5c4)n3)nc2)CC1] | 4-(3-(cyclopropylmethyl)-2- methyl-2H-indazol-5-yl)-N- (5-((4-ethylpiperazin-1- yl)methyl)pyridin-2-yl)-5- fluoropyrimidin-2-amine |
| 28 | 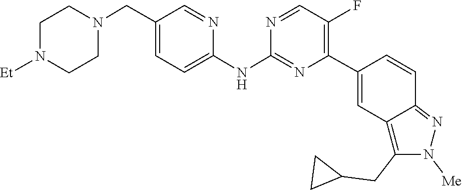 [SMILES: CCN1CCN(Cc2ccc(Nc3ncc(F)c(-c4ccc5nn(C)c(CC6CC6)c5c4)n3)nc2)CC1] | 4-(3-(cyclopropylmethyl)-7- fluoro-2-methyl-2H-indazol- 5-yl)-N-(5-((4-ethylpiperazin- 1-yl)methyl)pyridin-2-yl)-5- fluoropyrimidin-2-amine |
| 29 | 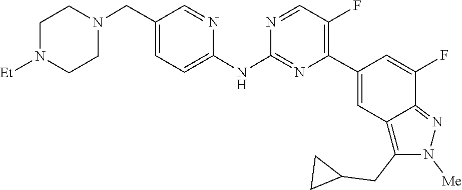 [SMILES: CCN1CCN(Cc2ccc(Nc3ncc(F)c(-c4cc(F)c5nn(C)c(CC6CC6)c5c4)n3)nc2)CC1] | 4-(3-cyclopropyl-2-ethyl-7- fluoro-2H-indazol-5-yl)-N-(5- ((4-ethylpiperazin-1- yl)methyl)pyridin-2- yl)pyrimidin-2-amine |
| 30 | 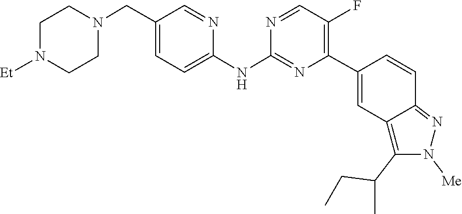 [SMILES: CCC(C)c1c2cc(-c3nc(Nc4ccc(CN5CCN(CC)CC5)cn4)ncc3F)ccc2nn1C] | 4-(3-(sec-butyl)-2-methyl- 2H-indazol-5-yl)-N-(5-((4- ethylpiperazin-1- yl)methyl)pyridin-2-yl)-5- fluoropyrimidin-2-amine |
| 31 | 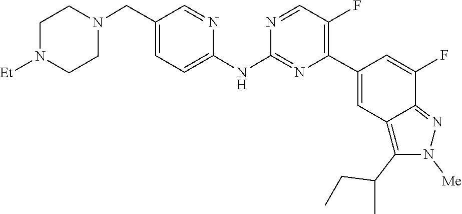 [SMILES: CCC(C)c1c2cc(-c3nc(Nc4ccc(CN5CCN(CC)CC5)cn4)ncc3F)cc(F)c2nn1C] | 4-(3-(sec-butyl)-7-fluoro-2- methyl-2H-indazol-5-yl)-N- (5-((4-ethylpiperazin-1- yl)methyl)pyridin-2-yl)-5- fluoropyrimidin-2-amine |
| 32 | 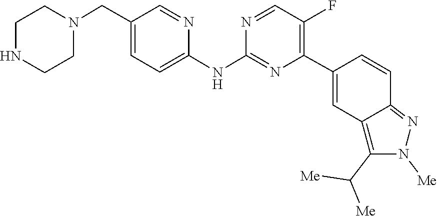 [SMILES: CC(C)c1c2cc(-c3nc(Nc4ccc(CN5CCNCC5)cn4)ncc3F)ccc2nn1C] | 5-fluoro-4-(3-isopropyl-2- methyl-2H-indazol-5-yl)-N- (5-(piperazin-1- ylmethyl)pyridin-2- yl)pyrimidin-2-amine |
| 33 | 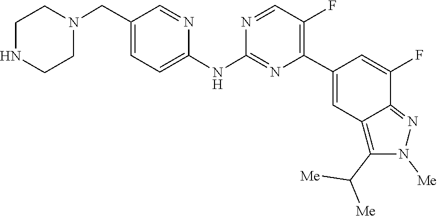 [SMILES: CC(C)c1c2cc(-c3nc(Nc4ccc(CN5CCNCC5)cn4)ncc3F)cc(F)c2nn1C] | 5-fluoro-4-(7-fluoro-3- isopropyl-2-methyl-2H- indazol-5-yl)-N-(5- (piperazin-1- ylmethyl)pyridin-2- yl)pyrimidin-2-amine |
| 34 | 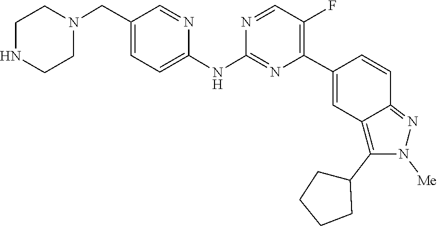 [SMILES: Cn1nc2ccc(-c3nc(Nc4ccc(CN5CCNCC5)cn4)ncc3F)cc2c1C1CCCC1] | 4-(3-cyclopentyl-2-methyl- 2H-indazol-5-yl)-5-fluoro-N- (5-(piperazin-1- ylmethyl)pyridin-2- yl)pyrimidin-2-amine |
| 35 | 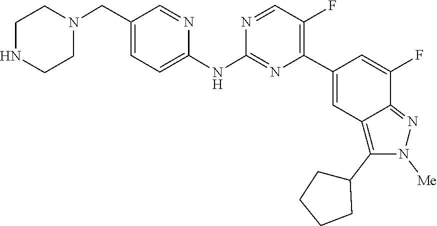 [SMILES: Cn1nc2c(F)cc(-c3nc(Nc4ccc(CN5CCNCC5)cn4)ncc3F)cc2c1C1CCCC1] | 4-(3-cyclopentyl-7-fluoro-2- methyl-2H-indazol-5-yl)-5- fluoro-N-(5-(piperazin-1- ylmethyl)pyridin-2- yl)pyrimidin-2-amine |
| 36 | 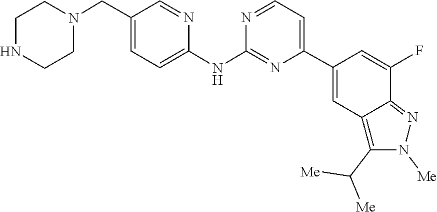 [SMILES: CC(C)c1c2cc(-c3ccnc(Nc4ccc(CN5CCNCC5)cn4)n3)cc(F)c2nn1C] | 4-(7-fluoro-3-isopropyl-2- methyl-2H-indazol-5-yl)-N- (5-(piperazin-1- ylmethyl)pyridin-2- yl)pyrimidin-2-amine |
| 37 | 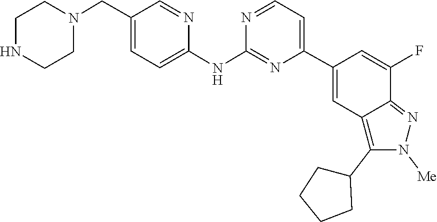 [SMILES: Cn1nc2c(F)cc(-c3ccnc(Nc4ccc(CN5CCNCC5)cn4)n3)cc2c1C1CCCC1] | 4-(3-cyclopentyl-7-fluoro-2- methyl-2H-indazol-5-yl)-N- (5-(piperazin-1- ylmethyl)pyridin-2- yl)pyrimidin-2-amine |
| 38 | 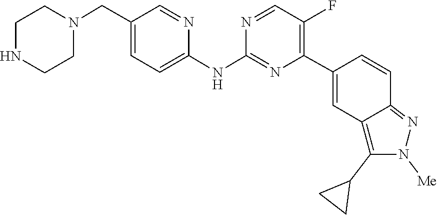 [SMILES: Cn1nc2ccc(-c3nc(Nc4ccc(CN5CCNCC5)cn4)ncc3F)cc2c1C1CC1] | 4-(3-cyclopropyl-2-methyl- 2H-indazol-5-yl)-5-fluoro-N- (5-(piperazin-1- ylmethyl)pyridin-2- yl)pyrimidin-2-amine |
| 36 | 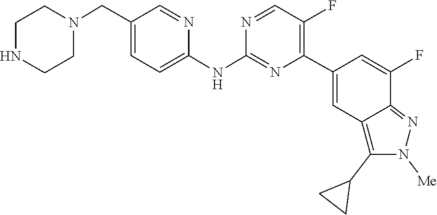 [SMILES: Cn1nc2c(F)cc(-c3nc(Nc4ccc(CN5CCNCC5)cn4)ncc3F)cc2c1C1CC1] | 4-(3-cyclopropyl-7-fluoro-2- methyl-2H-indaozl-5-yl)-5- fluoro-N-(5-(piperazin-1- ylmethyl)pyridin-2- yl)pyrimidin-2-amine |
| 40 | 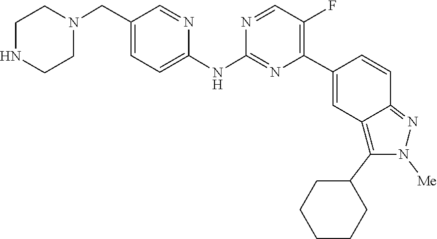 [SMILES: Cn1nc2ccc(-c3nc(Nc4ccc(CN5CCNCC5)cn4)ncc3F)cc2c1C1CCCCC1] | 4-(3-cyclohexyl-2-methyl- 2H-indazol-5-yl)-5-fluoro-N- (5-(piperazin-1- ylmethyl)pyridin-2- yl)pyrimidin-2-amine |
| 42 | 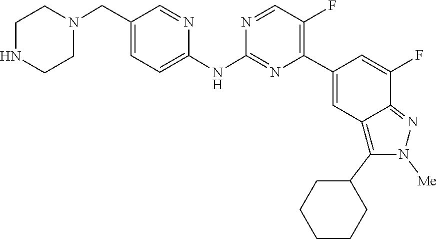 [SMILES: Cn1nc2c(F)cc(-c3nc(Nc4ccc(CN5CCNCC5)cn4)ncc3F)cc2c1C1CCCCC1] | 4-(3-cyclohexyl-7-fluoro-2- methyl-2H-indazol-5-yl)-5- fluoro-N-(5-(piperazin-1- ylmethyl)pyridin-2- yl)pyrimidin-2-amine |
| 42 | 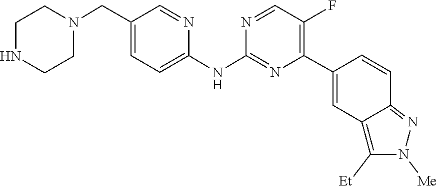 [SMILES: CCc1c2cc(-c3nc(Nc4ccc(CN5CCNCC5)cn4)ncc3F)ccc2nn1C] | 4-(3-ethyl-2-methyl-2H- indazol-5-yl)-5-fluoro-N-(5- (piperazin-1- ylmethyl)pyridin-2- yl)pyrimidin-2-amine |
| 43 | 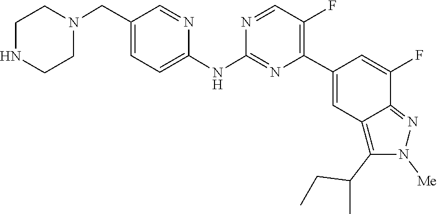 [SMILES: CCC(C)c1c2cc(-c3nc(Nc4ccc(CN5CCNCC5)cn4)ncc3F)cc(F)c2nn1C] | 4-(3-(sec-butyl)-7-fluoro-2- methyl-2H-indazol-5-yl)-5- fluoro-N-(5-(piperazin-1- ylmethyl)pyridin-2- yl)pyrimidin-2-amine |
| 44 | 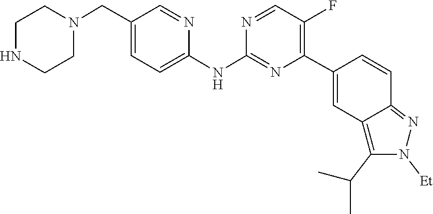 [SMILES: CCn1nc2ccc(-c3nc(Nc4ccc(CN5CCNCC5)cn4)ncc3F)cc2c1C(C)C] | 4-(2-ethyl-3-isopropyl-2H- indazol-5-yl)-5-fluoro-N-(5- (piperazin-1- ylmethyl)pyridin-2- yl)pyrimidin-2-amine |
| 45 | 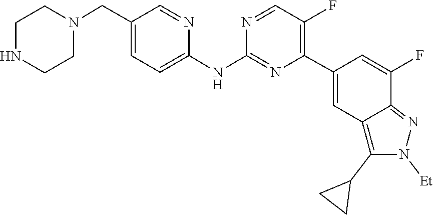 [SMILES: CCn1nc2c(F)cc(-c3nc(Nc4ccc(CN5CCNCC5)cn4)ncc3F)cc2c1C1CC1] | 4-(3-cyclopropyl-2-ethyl-7- fluoro-2H-indazol-5-yl)-5- fluoro-N-(5-(piperazin-1- ylmethyl)pyridin-2- yl)pyrimidin-2-amine |
| 46 | 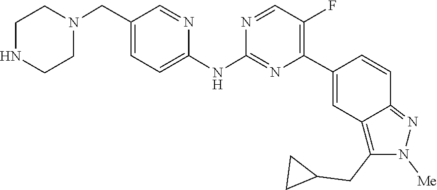 [SMILES: Cn1nc2ccc(-c3nc(Nc4ccc(CN5CCNCC5)cn4)ncc3F)cc2c1CC1CC1] | 4-(3-(cyclopropylmethyl)-2- methyl-2H-indazol-5-yl)-5- fluoro-N-(5-(piperazin-1- ylmethyl)pyridin-2- yl)pyrimidin-2-amine |
| 47 | 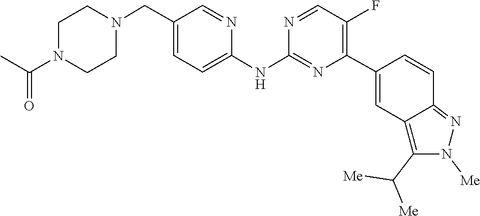 [SMILES: CC(=O)N1CCN(Cc2ccc(Nc3ncc(F)c(-c4ccc5nn(C)c(C(C)C)c5c4)n3)nc2)CC1] | 1-(4-((6-((5-fluoro-4-(3- isopropyl-2-methyl-2H- indazol-5-yl)pyrimidin-2- yl)amino)pyridin-3- yl)methyl)piperazin-1- yl)ethan-1-one |
| 48 | 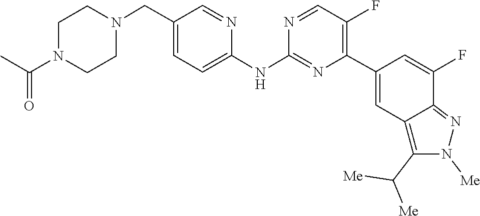 [SMILES: CC(=O)N1CCN(Cc2ccc(Nc3ncc(F)c(-c4cc(F)c5nn(C)c(C(C)C)c5c4)n3)nc2)CC1] | 1-(4-((6-((5-fluoro-4-(7- fluoro-3-isopropyl-2-methyl- 2H-indazol-5-yl)pyrimidin-2- yl)amino)pyridin-3- yl)methyl)piperazin-1- yl)ethan-1-one |
| 49 | 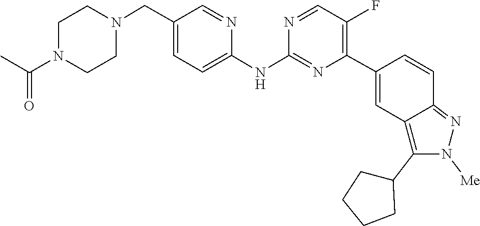 [SMILES: CC(=O)N1CCN(Cc2ccc(Nc3ncc(F)c(-c4ccc5nn(C)c(C6CCCC6)c5c4)n3)nc2)CC1] | 1-(4-((6-((4-(3-cyclopentyl-2- methyl-2H-indazol-5-yl)-5- fluoropyrimidin-2- yl)amino)pyridin-3- yl)methyl)piperazin-1- yl)ethan-1-one |
| 50 | 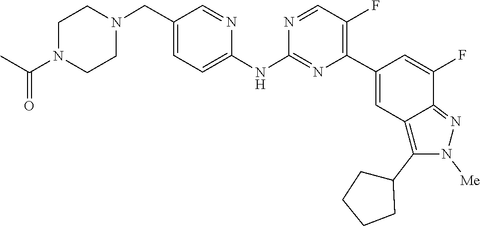 [SMILES: CC(=O)N1CCN(Cc2ccc(Nc3ncc(F)c(-c4cc(F)c5nn(C)c(C6CCCC6)c5c4)n3)nc2)CC1] | 1-(4-((6-((4-(3-cyclopentyl-7- fluoro-2-methyl-2H-indazol- 5-yl)-5-fluoropyrimidin-2- yl)amino)pyridin-3- yl)methyl)piperazin-1- yl)ethan-1-one |
| 51 | 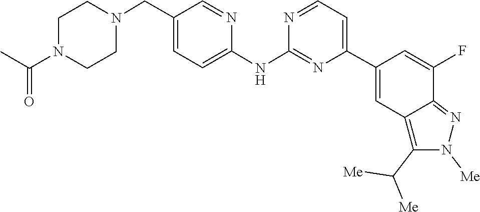 [SMILES: CC(=O)N1CCN(Cc2ccc(Nc3nccc(-c4cc(F)c5nn(C)c(C(C)C)c5c4)n3)nc2)CC1] | 1-(4-((6-((4-(7-fluoro-3- isopropyl-2-methyl-2H- indazol-5-yl)pyrimidin-2- yl)amino)pyridin-3- yl)methyl)piperazin-1- yl)ethan-1-one |
| 52 | 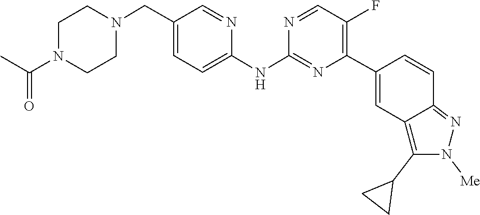 [SMILES: CC(=O)N1CCN(Cc2ccc(Nc3ncc(F)c(-c4ccc5nn(C)c(C6CC6)c5c4)n3)nc2)CC1] | 1-(4-((6-((4-(3-cyclopropyl- 2-methyl-2H-indazol-5-yl)-5- fluoropyrimidin-2- yl)amino)pyridin-3- yl)methyl)piperazin-1- yl)ethan-1-one |
| 53 | 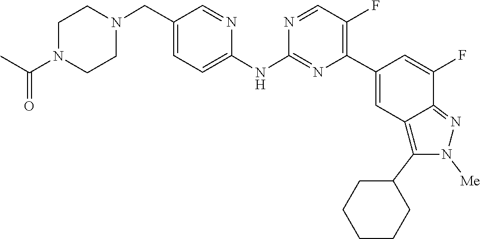 [SMILES: CC(=O)N1CCN(Cc2ccc(Nc3ncc(F)c(-c4cc(F)c5nn(C)c(C6CCCCC6)c5c4)n3)nc2)CC1] | 1-(4-((6-((4-(3-cyclohexyl-7- fluoro-2-methyl-2H-indazol- 5-yl)-5-fluoropyrimidin-2- yl)amino)pyridin-3- yl)methyl)piperazin-1- yl)ethan-1-one |
| 54 | 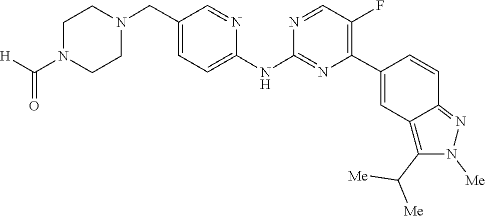 [SMILES: CC(C)c1c2cc(-c3nc(Nc4ccc(CN5CCN(C=O)CC5)cn4)ncc3F)ccc2nn1C] | 4-((6-((5-fluoro-4-(3- isopropyl-2-methyl-2H- indazol-5-yl)pyrimidin-2- yl)amino)pyridin-3- yl)methyl)piperazine-1- carbaldehyde |
| 55 | 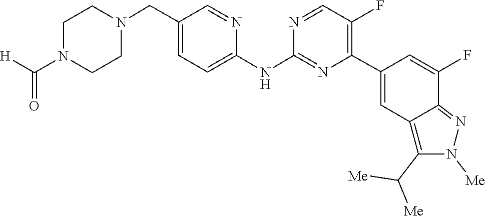 [SMILES: CC(C)c1c2cc(-c3nc(Nc4ccc(CN5CCN(C=O)CC5)cn4)ncc3F)cc(F)c2nn1C] | 4-((6-((5-fluoro-4-(7-fluoro- 3-isopropyl-2-methyl-2H- indazol-5-yl)pyrimidin-2- yl)amino)pyridin-3- yl)methyl)piperazine-1- carbaldehyde |
| 56 | 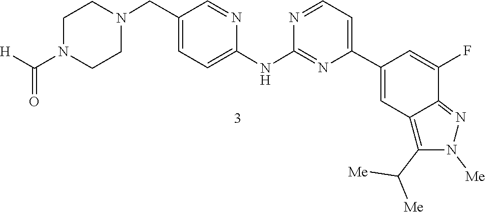 [SMILES: CC(C)c1c2cc(-c3ccnc(Nc4ccc(CN5CCN(C=O)CC5)cn4)n3)cc(F)c2nn1C] | 4-((6-((4-(7-fluoro-3- isopropyl-2-methyl-2H- indazol-5-yl)pyrimidin-2- yl)amino)pyridin-3- yl)methyl)piperazine-1- carbaldehyde |
| 57 | 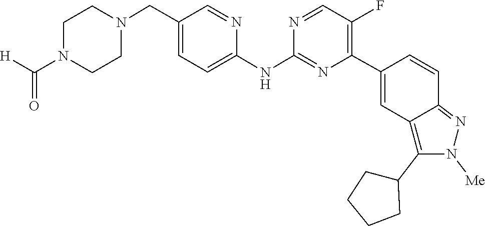 [SMILES: Cn1nc2ccc(-c3nc(Nc4ccc(CN5CCN(C=O)CC5)cn4)ncc3F)cc2c1C1CCCC1] | 4-((6-((4-(3-cyclopentyl-2- methyl-2H-indazol-5-yl)-5- fluoropyrimidin-2- yl)amino)pyridin-3- yl)methyl)piperazine-1- carbaldehyde |
| 58 | 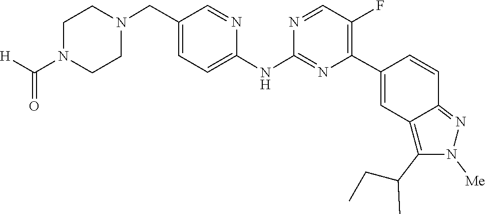 [SMILES: CCC(C)c1c2cc(-c3nc(Nc4ccc(CN5CCN(C=O)CC5)cn4)ncc3F)ccc2nn1C] | 4-((6-((4-(3-(sec-butyl)-2- methyl-2H-indazol-5-yl)-5- fluoropyrimidin-2- yl)amino)pyridin-3- yl)methyl)piperazine-1- carbaldehyde |
| 59 | 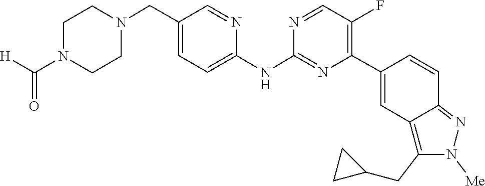 [SMILES: Cn1nc2ccc(-c3nc(Nc4ccc(CN5CCN(C=O)CC5)cn4)ncc3F)cc2c1CC1CC1] | 4-((6-((4-(3- (cyclopropylmethyl)-2- methyl-2H-indazol-5-yl)-5- fluoropyrimidin-2- yl)amino)pyridin-3- yl)methyl)piperazine-1- carbaldehyde |
| 60 | 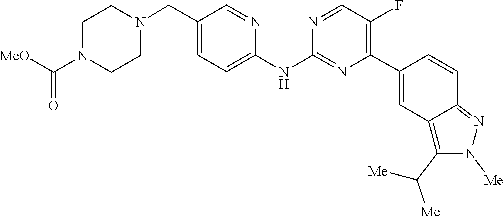 [SMILES: COC(=O)N1CCN(Cc2ccc(Nc3ncc(F)c(-c4ccc5nn(C)c(C(C)C)c5c4)n3)nc2)CC1] | methyl 4-((6-((5-fluoro-4-(3- isopropyl-2-methyl-2H- indazol-5-yl)pyrimidin-2- yl)amino)pyridin-3- yl)methyl)piperazine-1- carboxylate |
| 61 | 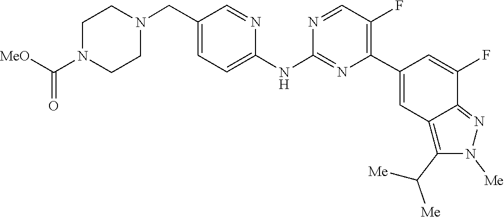 [SMILES: COC(=O)N1CCN(Cc2ccc(Nc3ncc(F)c(-c4cc(F)c5nn(C)c(C(C)C)c5c4)n3)nc2)CC1] | methyl 4-((6-((5-fluoro-4-(7- fluoro-3-isopropyl-2-methyl- 2H-indazol-5-yl)pyrimidin-2- yl)amino)pyridin-3- yl)methyl)piperazine-1- carboxylate |
| 62 | 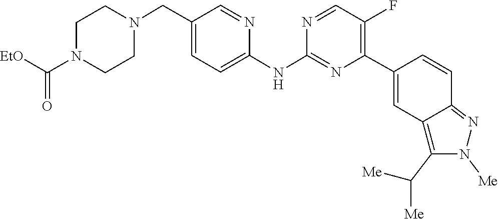 [SMILES: CCOC(=O)N1CCN(Cc2ccc(Nc3ncc(F)c(-c4ccc5nn(C)c(C(C)C)c5c4)n3)nc2)CC1] | ethyl 4-((6-((5-fluoro-4-(3- isopropyl-2-methyl-2H- indazol-5-yl)pyrimidin-2- yl)amino)pyridin-3- yl)methyl)piperazine-1- carboxylate |
| 63 | 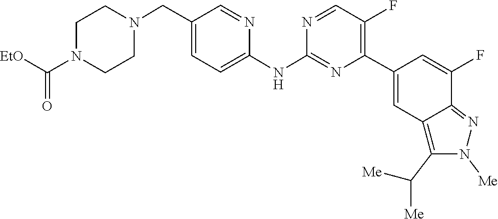 [SMILES: CCOC(=O)N1CCN(Cc2ccc(Nc3ncc(F)c(-c4cc(F)c5nn(C)c(C(C)C)c5c4)n3)nc2)CC1] | ethyl 4-((6-((5-fluoro-4-(7- fluoro-3-isopropyl-2-methyl- 2H-indazol-5-yl)pyrimidin-2- yl)amino)pyridin-3- yl)methyl)piperazine-1- carboxylate |
| 64 | 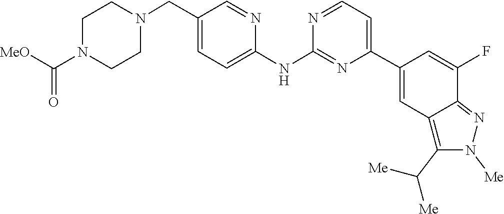 [SMILES: COC(=O)N1CCN(Cc2ccc(Nc3nccc(-c4cc(F)c5nn(C)c(C(C)C)c5c4)n3)nc2)CC1] | methyl 4-((6-((4-(7-fluoro-3- isopropyl-2-methyl-2H- indazol-5-yl)pyrimidin-2- yl)amino)pyridin-3- yl)methyl)piperazine-1- carboxylate |
| 65 | 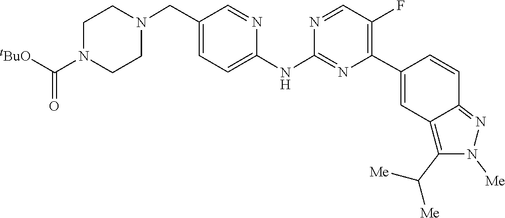 [SMILES: CC(C)c1c2cc(-c3nc(Nc4ccc(CN5CCN(C(=O)OC(C)(C)C)CC5)cn4)ncc3F)ccc2nn1C] | tert-butyl 4-((6-((5-fluoro-4- (3-isopropyl-2-methyl-2H- indazol-5-yl)pyrimidin-2- yl)amino)pyridin-3- yl)methyl)piperazine-1- carboxylate |
| 66 | 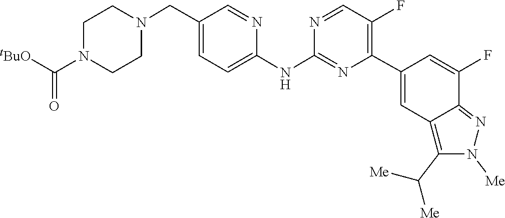 [SMILES: CC(C)c1c2cc(-c3nc(Nc4ccc(CN5CCN(C(=O)OC(C)(C)C)CC5)cn4)ncc3F)cc(F)c2nn1C] | tert-butyl 4-((6-((5-fluoro-4- (7-fluoro-3-isopropyl-2- methyl-2H-indazol-5- yl)pyrimidin-2- yl)amino)pyridin-3- yl)methyl)piperazine-1- carboxylate |
| 67 | 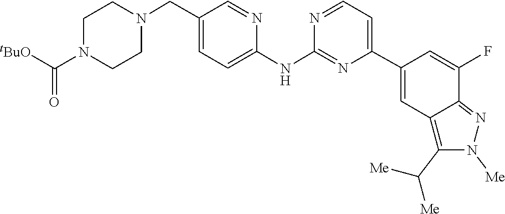 [SMILES: CC(C)c1c2cc(-c3ccnc(Nc4ccc(CN5CCN(C(=O)OC(C)(C)C)CC5)cn4)n3)cc(F)c2nn1C] | tert-butyl 4-((6-((4-(7-fluoro- 3-isopropyl-2-methyl-2H- indazol-5-yl)pyrimidin-2- yl)amino)pyridin-3- yl)methyl)piperazine-1- carboxylate |

### EXAMPLES

### Example 1. In Vivo Efficacy Studies in Mouse

### Materials and Methods

D-Luciferin (lot #0000204125) was obtained from Promega as a white powder and stored at −80° C. in a covered box to minimize light exposure. Saline was added to the D-luciferin powder to produce a clear yellow 15 mg/mi solution for in vivo imaging. D-Luciferin was prepared immediately prior to each bioluminescence imaging session and stored protected from light on wet ice during use.

Temozolomide (99.0% parent, MW 194 g/mol, FW 194 g/mol, 99% purity, C₆H₆N₆O₂, lot #S123705) was obtained from SelleckChem as a pink, fine powder. Upon receipt, it was stored protected from light at −20° C. The compound was formulated in a vehicle of sterile water. The dosing preparation was vortexed to form a clear, colorless, solution with a pH value of 6.3. The dosing solution was prepared weekly and stored at V° C. protected from light between treatments.

Compound 1 (92.8% parent, MW 489 g/mol, FW 525 g/mol, 99.7% purity, C₂₇H₃₃FN₈.HCl, was stored protected from light at 4° C. in a nitrogen rich environment. The compound was formulated in a vehicle of 10% ethanol, 10% CREMOPHOR®, and 80% saline (0.9% NaCl). The dosing preparation was prepared by first warming all vehicle components in a water bath set to approximately 42° C. The ethanol was added first to a sterile dosing vial containing pre-weighed BPI-1178 powder. The mixture was then vortexed to ensure that all powder was fully dissolved. Next, CREMOPHOR® was added to the solution and vortexed to mix. To finish, saline was added and the final mixture was vortexed to form a clear and colorless solution with a pH value of 5.7. The dosing solution was prepared fresh daily.

Abemaciclib (83.7% parent, MW 506 g/mol, FW 603 g/mol, 99.6% purity, C₂₇H₃₂F₂N₈.H₃CSO₃H, was obtained from Beta Pharma as a white, flakey powder. Upon receipt, it was stored protected from light at 4° C. in a nitrogen rich environment. The compound was formulated in a vehicle of 10% ethanol, 10% CREMOPHOR®, and 80% saline (0.9% NaCl). The dosing preparation was prepared by first warming all the vehicle components in a water bath set to approximately 42° C. The ethanol was added first to a sterile dosing vial containing pre-weighed abemaciclib powder. The mixture was then vortexed to ensure that all powder was fully dissolved. Next, CREMOPHOR® was added to the solution, which was vortexed to mix. To finish, saline was added and the final mixture vortexed to form a clear and colorless solution with a pH value of 4.0. The dosing solution was prepared fresh daily.

### Animals and Husbandry

Female Envigo Nude Mice (Hsd:Athymic Nude-Fox1^{nu}) were used in this study. They were 6-7 weeks old on Day 1 of the experiment. The animals were fed irradiated Harlan 2918.15 Rodent Diet and water ad libitum. Animals were housed in INNOVIVE® disposable ventilated caging with corn cob bedding inside BIOBUBBLE® Clean Rooms that provide H.E.P.A filtered air into the bubble environment at 100 complete air changes per hour. All treatments, body weight determinations, and tumor measurements were carried out in the bubble environment. The environment was controlled to a temperature range of 70°±2° F. and a humidity range of 30-70%.

All procedures were conducted in compliance with all laws, regulations and guidelines of the National Institutes of Health (NIH) and with the approval of Molecular Imaging, Inc.'s Animal Care and Use Committee. Molecular Imaging, Inc. is an AAALAC accredited facility.

### Example 1A. Cell Preparation

MG-Luc cells were obtained from ATCC. They were grown in Minimum Essential Medium (MEM) with Earle's Salts which was modified with 1% 100 mM Na pyruvate, 1% 100×NEAA (Non-Essential Amino Acids), 200 μg/mL G418 and supplemented with 10% non-heat-inactivated Fetal Bovine Serum (FBS) and 1% 100× Penicillin/Streptomycin/L-Glutamine (PSG). The growth environment was maintained in an incubator with a 5% CO₂ atmosphere at 37° C. When expansion was complete, the cells were trypsinized using 0.25% trypsin-EDTA solution. Following cell detachment, the trypsin was inactivated by dilution with complete growth medium and any clumps of cells were separated by pipetting. The cells were centrifuged at 200 rcf for 8 minutes at 4° C., the supernatant was aspirated, and the pellet was re-suspended in cold Dulbecco's Phosphate Buffered Saline (DPBS) by pipetting. An aliquot of the homogeneous cell suspension was diluted in a trypan blue solution and counted using a Luna automated cell counter. The cell suspension was centrifuged at 200 rcf for 8 minutes at 4° C. The supernatant was aspirated and the cell pellet was re-suspended in cold serum-free medium to generate a final concentration of 1.0E+08 trypan-excluding cells/ml. The cell suspension was maintained on wet ice during implantation. Following implantation, an aliquot of the remaining cells was diluted with a trypan blue solution and counted to determine the post-implantation cell viability.

| | Pre-implant | Post-implant |
|---|---|---|
| | viability (%) | viability (%) |
| Implant Day 1, Prep 1 | 95 | 95 |
| Implant Day 1, Prep 2 | 91 | 91 |
| Implant Day 2, Prep 1 | 96 | 98 |
| Implant Day 2, Prep 2 | 93 | 95 |
| Implant Day 3, Prep 1 | 92 | 95 |
| Implant Day 3, Prep 2 | 96 | 97 |

### Example 1B. Intracranial Implantation

Test mice were implanted intracranially on Days 0, 1, and 2 with 1.0E+06 cells per 10 μl. For aseptic surgical implantation, mice were injected with 0.2 mg/kg buprenorphine and anesthetized using 2% isoflurane in air. The mice were then secured in a stereotaxic frame (ASI instruments, Inc.) using non-rupture ear bars. Ocular ointment was applied to the eyes of the mice to prevent drying during surgery. A re-circulating 37° C. water heated pad was used to maintain the animal's body temperature during the implantation procedure.

Once in the stereotaxic frame, the cranium was swabbed with alternating chlorhexidine solution and 70% ethanol-saturated swabs to disinfect the skin surface and prepare for the incision. A 1 cm longitudinal incision was made centrally over bregma of the cranium using a #15 BD scalpel blade. The incision was retracted using small, serrated serrefines. The thin layer of connective tissue covering the surface of the skull was removed using dry cotton swabs under light pressure. Bleeding vessels were cauterized to prevent blood loss. A 0.9 mm drill bit was then centered over bregma, moved 2 mm right lateral, 1 mm anterior to the coronal suture and lowered to score the surface of the skull using the stereotaxic electrode manipulator arm. The drill was removed from the stereotaxic frame and the burr hole through the skull to the surface of the dura mater was completed by hand.

The cell suspension (stored on wet ice) was mixed thoroughly and drawn up into a 50 μl gas-tight Hamilton syringe. A standard 27 g needle was filled with the cell suspension to eliminate air pockets and the luer tip of the syringe was inserted into the needle hub. The syringe was secured to a custom-built syringe holder (ASI Instruments, Inc.) and attached to the stereotaxic frame manipulator arm. The syringe needle was centered over the burr hole and lowered until the beveled tip was level with the underside of the skull at the surface of the dura mater. The needle was then lowered 3 mm into the brain and retracted 1 mm to form a “reservoir” for the deposition of the cell suspension. 10 μl of the cell suspension (1×10⁶ cells/mouse) was then injected slowly into the brain tissue with any slight leakage (typical for IC implants) being absorbed with a dry cotton swab.

Following the injection, the needle was withdrawn and the burr hole was immediately sealed with bone wax to minimize the loss of implanted cells. The skull surface was then cleaned with alternating dry and 70% ethanol saturated cotton swabs to remove extraneous cells and deter extracranial tumor growth. The mouse was removed from the stereotaxic frame and the incision was closed using a stainless steel wound clip. Once the mouse regained consciousness and dorsal recumbancy, it was returned to its caging. Mice were implanted from Feb. 20-22, 2017.

### Example 1C. Treatment

All mice were sorted into study groups based on bioluminescence imaging (BLI) estimations of tumor burden. The mice were distributed to ensure that the mean tumor burden for all groups was within 10% of the overall mean tumor burden for the study population. As implants occurred over three days, Day 0 was defined as the middle implant date (Feb. 21, 2017). Treatment began on Day 21 for all groups regardless of initial implant date.
Group 1: Vehicle Control (10% EtOH, 10% CREMOPHOR®, 80% saline (0.9% NaCl)), 0.2 mL/20 g, PO, QD×21 (Days 21-41)
Group 2: Temozolomide, 6 mg/kg, PO, QD×5 (Days 21-25)
Group 3: Compound 1, 100 mg/kg, PO, QD×21 (Days 21-41)
Group 4: Abemaciclib, 100 mg/kg, PO, QD×21 (Days 21-41)
Group 5: Temozolomide, 6 mg/kg, PO, QD×5 (Days 21-25)+Compound 1, 100 mg/kg, PO, QD×21 (Days 21-41)
Group 6: Temozolomide, 6 mg/kg, PO, QD×5 (Days 21-25)+abemaciclib, 100 mg/kg, PO, QD×21 (Days 21-41)

### Example 1D. In Vivo Bioluminescence Imaging (BLI)

In vivo bioluminescence imaging (BLI) was performed using an IVIS Spectrum (Caliper Life Sciences, Hopkinton, Mass.). Animals were imaged up to 5 at a time under ca. 1-2% isoflurane gas anesthesia. Each mouse was injected subcutaneously with 150 mg/kg (15 mg/ml) D-luciferin and imaged in the prone position 10 minutes after the injection. Large binning of the CCD chip was used, and the exposure time was adjusted (2 seconds to 2 minutes) to obtain at least several hundred counts per image and to avoid saturation of the CCD chip. BLI images were collected on Days 21, 28, 35, 42, 49, 56, and 64.

Images were analyzed using Matlab R2015a software. Primary brain fixed-volume ROIs were placed on prone images for each individual animal to estimate brain tumor burden. Total flux (photons/sec) was calculated and exported for all ROIs to facilitate analyses between groups.

### Example 1E. Assessment of Side Effects

All animals were observed for clinical signs at least once daily. Animals were weighed on each day of treatment. Individual body weights were recorded 3 times weekly.

Treatment-related weight loss in excess of 20% is generally considered unacceptably toxic. For this study, a dosage level is described as tolerated if treatment-related weight loss (during and two weeks after treatment) is <20% and mortality during this period in the absence of potentially lethal tumor burdens is ≤10%.

Upon death or euthanasia, all animals were necropsied to provide a general assessment of potential cause of death and perhaps target organs for toxicity. The presence or absence of metastases was also noted. Remarkable observations of clinical signs and necropsy findings were recorded and individual and group toxicity findings were summarized.

### Example 2. Cell Permeability Study of Compound 1

### Summary

| | P-gp Substrate |
|---|---|
| Test Article | Classification |
| 1 | Negative |

The test article passed the lucifer yellow monolayer integrity test criteria (≤0.8×10⁻⁶ cm/s).

### Objectives

The objective of this study was to determine the P-gp substrate potential of one test article using MDR1-MDCK monolayers.

### Experimental Procedure

MDR1-MDCK cell monolayers were grown to confluence on collagen-coated, microporous membranes in 12-well assay plates. Details of the plates and their certification are shown below. The permeability assay buffer was Hanks' balanced salt solution (HBSS) containing 10 mM HEPES and 15 mM glucose at a pH of 7.4. The buffer in the receiver chamber also contained 1% bovine serum albumin. The dosing solution concentration was 5 μM of test article in the assay buffer+/−1 μM valspodar. Cells were first pre-incubated for 30 minutes with HBSS containing +/−1 μM valspodar. Cell monolayers were dosed on the apical side (A-to-B) or basolateral side (B-to-A) and incubated at 37° C. with 5% CO₂ in a humidified incubator. Samples were taken from the donor and receiver chambers at 120 minutes. Each determination was performed in duplicate. The flux of lucifer yellow was also measured post-experimentally for each monolayer to ensure no damage was inflicted to the cell monolayers during the flux period. All samples were assayed by LC-MS/MS using electrospray ionization. Analytical conditions are outlined in Appendix 1. The apparent permeability (Pₐₚₚ) and percent recovery were calculated as follows:

Pₐₚₚ=(dCᵣ/dt)×Vᵣ/(A×C_{A}) (1)

Percent Recovery=100×((Vᵣ×Cᵣ^{final})+(V_{d}×C_{d}^{final}))/(V_{d}×C_{N}) (2)
where
dC/dt is the slope of the cumulative concentration in the receiver compartment versus time in μM s⁻¹;
Vᵣ is the volume of the receiver compartment in cm³;
V_{d} is the volume of the donor compartment in cm³;
A is the area of the insert (1.13 cm² for 12-well);
C_{A} is the average of the nominal dosing concentration and the measured 120 minute donor concentration in μM;
C_{N} is the nominal concentration of the dosing solution in μM;
Cᵣ^{final} is the cumulative receiver concentration in μM at the end of the incubation period;
C_{d}^{final} is the concentration of the donor in μM at the end of the incubation period.

Efflux ratio (ER) is defined as Pₐₚₚ (B-to-A)/Pₐₚₚ (A-to-B).

### Cell Batch Quality Control Results

| Plates | 12-well | |
|---|---|---|
| Seed Date | Oct. 30, 2017 | |
| Passage Number | 17 | |
| Age at QC (days) | 7 | |
| Age at Experiment (days) | 8 | Acceptance Criteria |
| TEER Value (Ω*cm²) | 1591 | ≥1400 |
| Atenolol Pₐₚₚ, 10⁻⁶ cm/s | 0.06 | ≤0.5 |
| Propranolol Pₐₚₚ, 10⁻⁶ cm/s | 12.8 | 10-30 |
| Digoxin A-to-B Pₐₚₚ, 10⁻⁶ cm/s | 0.05 | ≤0.1 |
| Digoxin B-to-A Pₐₚₚ, 10⁻⁶ cm/s | 13.9 | none |
| Digoxin Efflux Ratio | 254 | ≥100 |

### Experimental Results

| | | | | | | | P-gp |
|---|---|---|---|---|---|---|---|
| | | Recov- | | | | | Substrate |
| Test | | ery | Pₐₚₚ (10⁻⁶ cm/s) | | | Efflux | Classifica- |
| Article | Direction | (%) | R1 | R2 | AVG | Ratio | tion |
| Compound | A-to-B | 22 | 5.25 | 5.43 | 5.34 | 1.5 | Negative |
| 1 | B-to-A | 51 | 7.14 | 8.40 | 7.77 | | |
| Compound | A-to-B | 19 | 5.12 | 4.05 | 4.59 | 1.0 | |
| 1 + 1 μM | B-to-A | 63 | 3.79 | 5.31 | 4.55 | | |
| Valspodar | | | | | | | |

### P-gp Substrate Classification Criteria:

ER≥2.0 without valspodar, and reduced by ≥50% with valspodar: Positive
ER≥2.0 without valspodar, and reduced by <50% with valspodar: Negative
ER<2.0 without and with valspodar: Negative
Based on the above results, Compound 1 is not a substrate for P-gp.

### Analytical Methods

### Liquid Chromatography

### Column: Waters ACQUITY UPLC® BEH Phenyl 30×2.1 mm, 1.7 μm

M.P. Buffer: 25 mM ammonium formate buffer, pH 3.5
Aqueous Reservoir (A): 90% water, 10% buffer
Organic Reservoir (B): 90% acetonitrile, 10% buffer
Flow Rate: 0.7 mL/minute

Gradient Program:

| Time (min) | % A | % B |
|---|---|---|
| 0.00 | 99 | 1 |
| 0.65 | 1 | 99 |
| 0.75 | 1 | 99 |
| 0.80 | 99 | 1 |
| 1.00 | 99 | 1 |

Total Run Time: 1.0 minute
Autosampler: 5 μL injection volume
Wash 1: water/methanol/2-propanol:1/1/1; with 0.2% formic acid
Wash 2: 0.1% formic acid in water

### Mass Spectrometry

### Instrument: PE SCIEX API 4000

### Interface: Turbo Ionspray

Mode: Multiple reaction monitoring
Method: 1.0 minute duration

Settings:

| Test Article | +/− | Q1 | Q3 | DP | EP | CE | CXP | IS |
|---|---|---|---|---|---|---|---|---|
| BPI-1178-7 | + | 489.4 | 375.3 | 12 | 10 | 28 | 12 | 5500 |

### TEM: 500; CAD: 7; CUR: 30; GS1: 50; GS2: 50

### Example 3. Brain Concentration and Brain/Plasma Ratio in Mouse

Mice were dosed at 10 mg/kg p.o. As shown in Tables 1 and 2, brain concentration of Compound 1 was observed to be approximately 3-fold higher than that of abemaciclib, and the brain/plasma (B/P) ratio for Compound 1 was 1.43 vs. only 0.43 for abemaciclib.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. The term “about” generally includes up to plus or minus 10% of the indicated number. For example, “about 10%” may indicate a range of 9% to 11%, and “about 20” may mean from 18 to 22. Preferably “about” includes up to plus or minus 6% of the indicated value. Alternatively, “about” includes up to plus or minus 5% of the indicated value. Other meanings of “about” may be apparent from the context, such as rounding off, so, for example “about 1” may also mean from 0.5 to 1.4.

All publications cited herein are incorporated by reference in their entirety for all purposes. It should be understood that embodiments described herein should be considered as illustrative only, without limiting the scope of the invention. Descriptions of features or aspects within each embodiment should typically be considered as available for other similar features or aspects in other embodiments.

While several embodiments have been described in the Examples above, it will be understood by those of ordinary skill in the art that various changes in form and details may be made therein without departing from the spirit and scope of the disclosure as defined by the following claims.

1. A method of treating a brain cancer or brain metastasis of another cancer, or preventing brain metastasis in a subject with another cancer, comprising administering to a subject in need thereof, a therapeutically effective amount of a composition comprising a compound of formula (I): 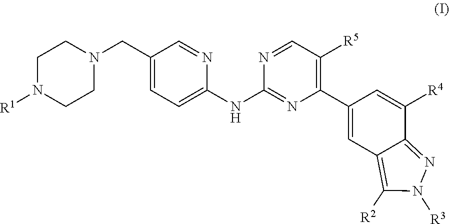
or a pharmaceutically acceptable salt, solvate, or prodrug thereof,
wherein:
R¹ is hydrogen, C₁-C₈ alkyl, C₃-C₇ cycloalkyl, R⁶C(O)—, or R⁷O(CO)—;
R² and R³ are each independently hydrogen, C₁-C₈ alkyl, C₃-C₇ cycloalkyl, or C₃-C₇ cycloalkylmethyl;
R⁴ is hydrogen, halogen, C₁-C₈ alkyl, or C₃-C₇ cycloalkyl;
R⁵ is hydrogen or halogen;
R⁶ is hydrogen, C₁-C₈ alkyl; or C₃-C₇ cycloalkyl; and
R⁷ is C₁-C₈ alkyl; or C₃-C₇ cycloalkyl,
wherein any said alkyl or cycloalkyl is optionally substituted.

2. The method of claim 1, wherein R¹ is hydrogen or C₁-C₆ alkyl.

3. The method of claim 1, wherein R¹ is methyl, ethyl, propyl, or isopropyl.

4. The method of claim 1, wherein R² is C₁-C₆ alkyl, C₃-C₆ cycloalkyl, or C₃-C₆ cycloalkylmethyl.

5. The method of claim 1, wherein R² is methyl, ethyl, propyl, isopropyl, cyclopropyl, cyclopentyl, cyclopropylmethyl, or cyclopentylmethyl.

6. The method of claim 1, wherein R³ is C₁-C₆ alkyl or C₃-C₆ cycloalkyl.

7. The method of claim 1, wherein R³ is methyl, ethyl, propyl, isopropyl, or cyclopropyl.

8. The method of claim 1, wherein R⁴ is hydrogen or halogen.

9. The method of claim 1, wherein R⁵ is hydrogen or fluoro.

10. The method of claim 1, wherein R¹ is hydrogen, methyl, or ethyl; R² is isopropyl, cyclopropyl, cyclopropylmethyl, or cyclopentyl; R³ is methyl or ethyl; R⁴ is hydrogen or fluoro; and R⁵ is hydrogen or fluoro.

11. The method of claim 1, wherein the compound of formula (I) is selected from the group consisting of the compounds listed in Table 3.

12. A method of treating a brain cancer or brain metastasis of another cancer, or preventing brain metastasis in a subject with another cancer, comprising administering to a subject in need thereof, a therapeutically effective amount of a composition comprising a compound of formula: 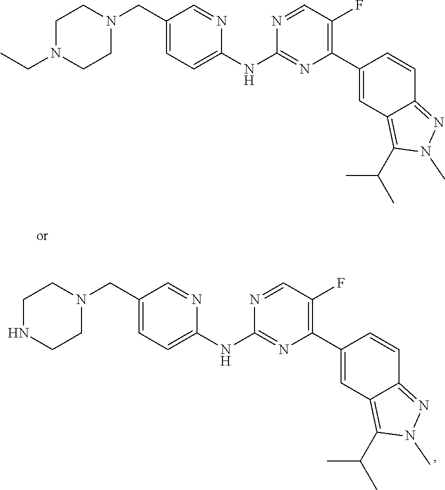 [SMILES: CCN1CCN(Cc2ccc(Nc3ncc(F)c(-c4ccc5nn(C)c(C(C)C)c5c4)n3)nc2)CC1 ; CC(C)c1c2cc(-c3nc(Nc4ccc(CN5CCNCC5)cn4)ncc3F)ccc2nn1C]
or a pharmaceutically acceptable salt, solvate, or prodrug thereof.

13. The method of claim 12, wherein the brain cancer or another cancer expresses CDK4 and/or CDK6.

14. The method of claim 12, wherein the brain cancer is glioblastoma.

15. The method of claim 12, wherein the another cancer is selected from the group consisting of breast cancers, lung cancers, especially non-small cell lung cancer (NSCLC), colorectal cancers, prostate cancer, kidney cancer, melanomas, mantel cell lymphoma (MCL), chronic myeloid leukemia (CML), and acute myeloid leukemia (AML).

16. The method of claim 12, wherein the administering is in conjunction with administration to the subject a second therapeutic agent.

17. The method of claim 16, wherein the second therapeutic agent is a different CDK inhibitor, a HER2 inhibitor, an mTOR inhibitor, or an EGFR inhibitor.

18. (canceled)

19. A method of treating a brain cancer or brain metastasis from another cancer, or prevention of brain metastasis in a subject with another cancer, associated with CDK4 and/or CDK6 activity, comprising administering to a subject in need thereof a compound of the formula: 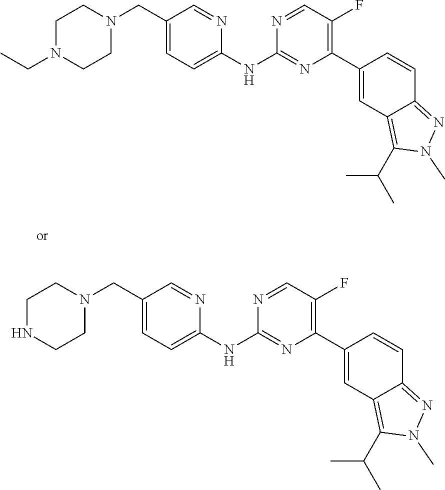 [SMILES: CCN1CCN(Cc2ccc(Nc3ncc(F)c(-c4ccc5nn(C)c(C(C)C)c5c4)n3)nc2)CC1 ; CC(C)c1c2cc(-c3nc(Nc4ccc(CN5CCNCC5)cn4)ncc3F)ccc2nn1C]
or a pharmaceutically acceptable salt, solvate, or prodrug thereof.

20. The method of claim 19, wherein the brain cancer is glioblastoma.

21. The method of claim 19, wherein the another cancer is selected from the group consisting of breast cancers, lung cancers (e.g., non-small cell lung cancer (NSCLC)), colorectal cancers, prostate cancer, kidney cancer, melanomas, mantel cell lymphoma (MCL), chronic myeloid leukemia (CML), and acute myeloid leukemia (AML).
